(19) United States
(12) Patent Application Publication
(10) Pub. No.: US 2006/0088545 A1
(43) Pub. Date: Apr. 27, 2006

(54) Use of inhibitors of the protease of the human immunodeficiency virus (hiv) to block cell migration and/or invasion, tissue infiltration and oedema for the therary of diseases associated therewith

(75) Inventor: Barbara Ensoli, Rome (IT)

Correspondence Address: JONES DAY, 222 EAST 41ST ST, NEW YORK, NY, 10017, US

(73) Assignee: INSTITUTO SUPERIORE DI SANITA, Rome (IT)

(21) Appl. No.: 10/549,958

(22) PCT Filed: Apr. 18, 2002
(86) PCT No.: PCT/EP02/04303

§ 371 Date: Sep. 15, 2005

(30) Foreign Application Priority Data
Apr. 18, 2001 (IT) RM2001A000210

(51) Int. Cl. A61K 31/551; A61K 39/21; A61K 31/522

(52) U.S. Cl. 424/188.1; 514/220; 514/263.34

(57) ABSTRACT

The present invention relates to a method to block the invasion of normal, neoplastic inflammatory or immune cells, tissue infiltration, and/or oedema formation through inhibition or modulation of molecules and proteolytic enzymes such as—but not exclusively—MMPs, for the therapy of all diseases whose pathogenesis is related to the above processes, including tumours, non-neoplastic angioproliferative diseases, inflammatory diseases, or autoimmune diseases, the method being based on the use of inhibitors of the protease of the HIV virus (HIV-PI).

### FIELD OF THE INVENTION

The present invention refers to the use of inhibitors of the protease of the human immunodeficiency virus (HIV) to inhibit invasion of tissues by normal and/or neoplastic cells, for the therapy of diseases associated therewith, such as Kaposi's sarcoma, tumours, angioproliferative, inflammatory or autoimmune diseases, associated or not with HIV infection.

### PRIOR ART

The inhibitors of the protease of the HIV virus are compounds with a known anti-retroviral activity that are described, for example, in Deeks et al. (Deeks et al., 1997). They are used in the therapy of HIV infection in subjects affected by the acquired immuno-deficiency syndrome (AIDS) with the function of inhibiting the maturation of the virus and blocking its replication (Deeks et al., 1997). In this description the inhibitors of the protease of the HIV virus will also be indicated below as HIV-PI.

Kaposi's sarcoma (KS) is a tumour associated with infection by the human herpesvirus 8 (HHV8) and is particularly frequent in subjects infected with the HIV virus (AIDS-KS) (Ensoli and Stürzl, 1998). KS is also observed in subject not infected with HIV, particularly in the Mediterranean area and in Italy (classic KS), in Africa (endemic KS) and in organ-transplanted individuals subjected to immuno-suppressive therapy (iatrogenic KS) (Ensoli and Stürzl, 1998). The deregulation of the immune system seems to be a necessary condition for the development of KS in subjects infected with HHV8 (Ensoli and Stürzl, 1998).

Various authors have described a reduced incidence of KS and of lymphomas (International Collaboration on HIV and Cancer, 2000) or regression (Lebbé et al., 1998; Cattelan et al., 1999) of KS in patients infected with HIV and treated with combinations of anti-retroviral drugs containing at least one HIV-PI (Deeks et al., 1997). KS is a vascular tumour characterised by angiogenesis, vascular ells), and infiltration of inflammatory cells; and is particularly frequent and aggressive in homosexual and bisexual males co-infected by HIV and HHV-8 (Ensoli and Stürzl, 1998). The formation of the lesions is mediated by cytokines and chemokines with angiogenic, proliferative, oedemigenic, chemotactic and inflammatory effects. These cytokines are produced by KS cells, by activated endothelial cells and by immune cells infiltrating the tissues (Ensoli et al., 1989; Ensoli et al., 1994a; Ensoli et al., 1994b; Fiorelli et al., 1995; Samaniego et al., 1995; Samaniego et al., 1997; Samaniego et al., 1998; Barillari et al., 1999a). Among the angiogenic factors, the basic fibroblast growth factor (bFGF) is expressed at high levels in KS lesions and is the most important autocrine and paracrine factor for KS growth and angiogenesis (Ensoli et al., 1989; Ensoli et al., 1994a; Ensoli et al., 1994b; Samaniego et al., 1995; Fiorelli et al., 1995; Samaniego et al., 1997; Samaniego et al., 1998; Barillari et al., 1999a). In fact, antibodies or anti-sense oligomers directed against bFGF block both angiogenesis and KS-like lesions development induced by the inoculation of primary KS cells in nude mice, and in vitro growth of KS cells (Ensoli et al., 1989; Ensoli et al., 1994b; Barillari et al., 1999b). Vice versa, the inoculation of bFGF in nude mice promotes the development of KS-like angioproliferative lesions (Ensoli et al., 1994a; Samaniego et al., 1998; Barillari et al., 1999a), the frequency and aggressiveness of which are increased by the protein Tat of HIV-1, which is able to mimic the action of proteins of the extracellular matrix. In particular, to act on the KS, Tat requires the presence of bFGF or inflammatory cytokines which, in turn, induce in endothelial cells and KS cells bFGF production and expression of integrins that act as receptors for Tat (Ensoli et al., 1990; Barillari et al., 1992; Barillari et al., 1993; Ensoli et al., 1994a; Albini et al., 1995; Fiorelli et al., 1995; Fiorelli et al., 1998; Fiorelli et al., 1999; Barillari et al., 1999a and 1999b). Another inducer of growth, angiogenesis and vascular permeability present in KS is the vascular endothelial growth factor (VEGF), which co-operates with bFGF in the angiogenesis and oedema of KS (Samaniego et al., 1998). Other factors present in KS and which co-operate in its formation are interleukin (IL)-1, IL-6. the tumour necrosis factor (TNF)α, interferon (IFN)γ, the granulocyte-monocyte colony-stimulating factor (GM-CSF), the platelet-derived growth factor (PDGF), oncostatin-M and chemokines (RANTES, MIP-1α, MIP-1β, and others) (Ensoli and Stürzl, 1998). In particular, the inflammatory cytokines such as IL-1, IL-6, TNFα and IFNγ induce KS cells and endothelial cells to produce bFGF and VEGF, induce endothelial cells to acquire the phenotype of KS cells, to become angiogenic in vivo, and induce KS lesions in mice (Samaniego et al., 1995; Fiorelli et al., 1995; Fiorelli et al., 1998; Barillari et al., 1999a). In addition to promoting the growth of KS, bFGF, like VEGF, is able to activate all the processes that are required for angiogenesis. Angiogenesis in turn is fundamental for the growth and metastasis of tumours and for non-neoplastic angioproliferative diseases and is often an important component in chronic inflammatory diseases (Carmeliet and Jain, 2000). In addition, chemokines produced by activated endothelial cells, KS cells, and inflammatory cells infiltrating tissues, such as RANTES, MIP-1α, MIP-1β, IL-8, MCP-1 and others have indirect angiogenic effects and act as chemoattractants for inflammatory cells, thus inducing a further recruitment and infiltration of inflammatory and immune cells (infected or not by HHV-8) in tissues and lesions (Ensoli and Stürzl, 1998). In this context, angiogenesis, infiltration of tissues by inflammatory cells, and oedema all require the degradation of the vascular basal membrane and/or extracellular matrix by specific proteases allowing the directional migration of cells in the perivascular space (invasion and migration of endothelial or inflammatory/immune cells), or favouring the efflux of fluids from the bloodstream (Carmeliet and Jain, 2000). In addition, angiogenesis requires a third step consisting in the proliferation of endothelial cells.

In particular, the degradation of the vascular basal membrane and interstitial tissue is mediated by the metalloproteases of the matrix (MMP). The MMPs themselves are necessary for tumour and metastatic growth, for the infiltration of tissues by inflammatory cells and for oedema formation (Stetler-Stevenson, 1999). In particular, infiltration of tissues by inflammatory cells has an important role in cancer, inflammation, and autoimmune diseases as these cells produce factors, including angiogenic factors and inflammatory cytokines, with paracrine actions on neighbourhood cells. Among MMPs, MMP-2 is essential for angiogenesis, it is induced by bFGF and is strongly expressed in primary lesions of KS and in other neoplasias (Ensoli et al., 1994a; Barillari et al., 1999b; Stetler-Stevenson, 1999), whereas MMP-9 is the most important MMP mediating infiltration of monocytes and lymphocytes in tissues. The inhibition of the migration, invasion or proliferation of endothelial cells, or of the activity of MMP-2 and MMP-9 or other MMPs, is able to block angiogenesis and constitutes the rationale for the anti-angiogenic and anti-tumour therapies currently in use (Stetler-Stevenson, 1999; Koivunen et al., 1999; Carmeliet and Jain, 2000). Moreover, local and systemic MMP concentration and/or activity show significant changes in several pathological conditions including infections, multiple sclerosis, inflammatory diseases, immune diseases, and cancer (Fujimoto et al., 1993; Leppert D et al., 1998; Leppert et al., 2000;) and can therefore be targeted for diagnosis, prognosis and therapy of these diseases.

The lower incidence and the regression of KS observed in individuals infected with HIV and treated with HIV-PI (Lebbé et al., 1998; Cattelan et al., 1999; International Collaboration on HIV and Cancer, 2000) has been related to this drug capabity of inhibiting the replication of HIV and, consequently, the production and release of the protein Tat of HIV-1, a powerful KS progression factor (Ensoli et al., 1990; Ensoli et al., 1994a; Barillari et al., 1999a; Barillari et al., 1999b). Moreover, by reconstituting the number and the function of specific cytotoxic T lymphocytes and the natural killer activity, the treatment with HIV-PI increases a protective immune response against HHV-8, the virus considered to be the cause of KS (Ensoli and Stürzl, 1998). In fact, subjects treated with HIV-PI show a reduction of both of HIV (Deeks et al., 1997) and HHV-8 load, and the reappearance of the immunological responses against HHV-8 (Blum et al., 1997; Rizzieri et al., 1997; Lebbé et al., 1998; Osman et al., 1999; Sirianni et al., 1999; Sirianni et al., 2000; Wang et al., 2000).

In this context, recent data indicated that HIV-PI modulate dendritic cell function and antigen presentation (André et al, 1998, Gruber et al, 2001), reducing T cell activation and inflammatory cytokines production also in the absence of HIV (Tovo, AIDS 2000, Ledru et al, 2000). Further, one HIV-PI, namely ritonavir, has been shown to have profound effects on proteasome activity, resulting in altered antigenic epitope processing and presentation by major histocompatibility complex (MHC) class I (André et al., PNAS, 1998; patent appl.n WO99/63998). These data have indicated that HIV-PI may have immunomodulating properties (André et al., PNAS, 1998; Tovo, AIDS 2000; patent appl.n WO99/63998, patent appl.n WO0033654). Moreover, since proteasome is also known to be involved in angiogenesis (Oikawa et al., 1998), these data suggested that HIV-PI may also affect angiogenesis. In addition, recent data indicated that HIV-PI have modulating properties for several cell processes including cell activation, survival and proliferation (patent appl.n WO99/63998, patent appl.n WO0033654). On the basis of these data, it has been suggested that protease inhibitors, including HIV-PI, inhibitors of proteasome, microbial and viral protease inhibitors, and cystein or serin protease inhibitors, may be used to modulate cell responses and metabolism for the therapy of a variety of human diseases by acting on these cell responses.

By contrast, we hypothesised that HIV-PI could have direct and specific effects on cellular invasion and vascular permeability due to activities on enzymes or molecules involved in these processes; namely, molecules which are not related to the cell proteasome such as (but not exclusively) MMPs. Therefore, the lower incidence and regression of KS observed in individuals treated with HIV-PI could be due to inhibition by HIV-PI of endothelial and KS cell invasion, infiltration of tissues by inflammatory and/or immune cells, and oedema formation. It should be pointed out that these effects of HIV-PI could not be anticipated on the basis of the existing studies. In fact, all the studies agree in attributing the lower incidence or regression of KS in subjects treated with HIV-PI to the inhibition of the HIV infection with consequent reduction of the expression of the protein Tat, to the reconstruction of the immune system and the consequent disappearance of HHV8 from blood or lesions, or to immuno-modulatory effects of HIV-PI (Blum et al., 1997; Rizzieri et al., 1997; Lebbé et al., 1998; De Milito et al., 1999; Cattelan et al., 1999; Osman et al., 1999; Sirianni et al., 1999; Sirianni et al., 2000; Wang et al., 2000; patent appl.n WO99/63998, patent appl.n WO0033654). On the contrary, the effects of HIV-PI that we hypothesised have never been described or studied before.

### SUMMARY OF THE INVENTION

It is an object of the present invention the use of the inhibitors of the protease of the HIV virus (HIV-PI) to block the migration and/or invasion of normal, neoplastic inflammatory or immune cells, tissue infiltration, and/or oedema formation through inhibition or modulation of molecules and proteolytic enzymes such as—but not exclusively—MMPs, for the therapy of all diseases whose pathogenesis is related to the above processes, including tumours, non-neoplastic angioproliferative diseases, inflammatory diseases, or autoimmune diseases.

Another object of the invention is the method to block the migration and/or invasion of normal, neoplastic, inflammatory or immune cells, tissue infiltration, and/or oedema formation through inhibition or modulation of molecules and proteolytic enzymes such as—but not exclusively—MMPs, obtained by the use of the inhibitors of the protease of the HIV virus (HIV-PI).

Another object of the present invention the use of the inhibitors of the protease of the HIV virus (HIV-PI) to produce drugs endowed with the ability to block cell migration and/or invasion and tissue infiltration through the inhibition of molecules and proteolytic enzymes such as—but not exclusively—MMPs, to elicit an anti-angiogenic action for the treatment of tumours and non-neoplastic angioproliferative diseases in subjects infected or not infected with the HIV virus.

Another object of the invention is the use of the inhibitors of the protease of the HIV virus (HIV-PI) to block tumour cell invasion in subjects infected or not infected with the HIV virus.

Another object of the invention is the use of the inhibitors of the protease of the HIV virus (HIV-PI) to produce drugs with an anti-oedemigenic activity and capable of blocking infiltration of tissues by inflammatory and immune cells for the therapy of inflammatory and autoimmune diseases in subjects infected or not infected with the HIV virus.

Another object of the invention is the use of the inhibitors of the protease of the HIV virus (HIV-PI) to produce drugs for the treatment of Kaposi's sarcoma in subjects infected or not infected with the HIV virus.

Another object of the invention is the use of the inhibitors of the protease of the HIV virus (HIV-PI) to produce drugs endowed with the ability to block cell migration and/or invasion and tissue infiltration through the inhibition of molecules and proteolytic enzymes such as—but not exclusively—MMPs, to elicit an anti-angiogenic, anti-tumour, anti-oedemigenic and/or anti-inflammatory action for the treatment of Kaposi's sarcoma, tumours and non-neoplastic angioproliferative, inflammatory and autoimmune diseases in subjects infected with the HIV virus.

A further object of the invention is the use for the above purposes of the compounds known as Crixivan® (indinavir) marketed by Merck, Sharp and Dohme; Invirase® or Fortovase® (saquinavir), marketed by Roche; Norvir® (ritonavir), marketed by Abbott Laboratories; Viracept® (nelfinavir), marketed by Roche; Agenerase® (amprenavir), marketed by Glaxo Wellcome; Kaletra® (lopinavir and ritonavir), marketed by Abbott Laboratories.

Another object of the invention is the use of the inhibitors of the protease of the HIV virus (HIV-PI) and of the compounds listed above for the above indications in combination with one another and/or in association with anti-inflammatory, anti-angiogenic or anti-tumour drugs.

A further object of the invention is the use of those chemical analogues or derivatives) of the inhibitors of the protease of HIV, HIV-PI, listed above with the capability of blocking the invasion of normal, neoplastic, inflammatory or immune cells and tissue infiltration, due to inhibition of molecules and proteolytic enzymes such as—but not exclusively—MMPs, and thus endowed with anti-angiogenic, anti-tumour, anti-oedemigenic and anti-inflammatory activity, alone or combined with one another and/or in association with anti-inflammatory, anti-angiogenic or anti-tumour drugs.

Further objects of the invention will be evident from the following detailed description of the invention.

### BRIEF DESCRIPTION OF THE FIGURES

FIG. 1 (panels A e B). Indinavir and saquinavir have no effect on the basal or bFGF-induced proliferation of primary macrovascular (humbelical vein) endothelial cells. Panel A: effect of indiinavir on basal or bFGF-induced cell proliferation; Panel B: effect of saquinavir on basal or bFGF-induced cell proliferation.

FIG. 2 (panels A and B). Indinavir and saquinavir inhibit the migration of macrovascular (umbilical vein) endothelial cells in response to bFGF. Panel A. effect of indinavir on cell migration; Panel B: effect of saquinavir on cell migration.

FIG. 3 (panels A and B). Indinavir and saquinavir inhibit the invasion of macrovascular (umbilical vein) endothelial cells in response to bFGF. Panel A: effect of indinavir on cell invasion; Panel B: effect of saquinavir on cell invasion.

FIG. 4. Indinavir and saquinavir do not interfere with the proliferation of microvascular (dermal) endothelial cells in response to bFGF.

FIG. 5. Indinavir and saquinavir inhibit the invasion of microvascular (dermal) endothelial cells in response to bFGF.

FIG. 6. Indinavir and saquinavir do not interfere with the proliferation of smooth muscle cells in response to bFGF.

FIG. 7. Indinavir and saquinavir inhibit the invasion of smooth muscle cells in response to bFGF.

FIG. 8 (panels A, B and C). Indinavir blocks the activation of MMP-2. Panel A: gelatinolytic activity corresponding to latent MMP-2 (72 kD), pre-active MMP-2 (64 kD) or active MMP-2 (62 kD) in supernatants form endothelial cells treated or not with bFGF in the presence or absence of indinavir; Panel B: densitometric quantitation of latent MMP-2; Panel C: densitometric quantitation of pre-active and active MMP-2 forms.

FIG. 9 (panels A, B and C). Saquinavir blocks the activation of MMP-2. Panel A: gelatinolytic activity corresponding to latent MMP-2 (72 kD), pre-active MMP-2 (64 kD) or active MMP-2 (62 kD) in supernatants form endothelial cells treated or not with bFGF in the presence or absence of saquinavir; Panel B: densitometric quantitation of latent MMP-2; Panel C: densitometric quantitation of preactive and active MMP-2 forms.

FIG. 10. Indinavir and saquinavir block the autoproteolytic conversion of pre-MMP-2 to its active form.

FIG. 11 (panels A and B). Saquinavir blocks the production of casein-specific MMP in endothelial cells. Panel A: casein zymography of supernatants from endothelial cells trated with bFGF or TPA in the presence or absence of saquinavir for 8 hours; Panel B: casein zymography of supernatants from endothelial cells trated with bFGF or TPA in the presence or absence of saquinavir for 24 hours;

FIG. 12 [(1) (panels a-d) and (2) (panels a-h)]. Indinavir and saquinavir inhibit the formation of angioproliferative lesions induced by bFGF in the nude mouse. A) panel a: injection sites from a representative mice treated with saline solution and inoculated with matrigel alone; panel b: injection sites from a representative mice treated with saline and inoculated with bFGF in matrigel; panel c: injection sites from a representative mice treated with indinavir and inoculated with bFGF in matrigel; panel d: injection sites from a representative mice treated with saquinavir and inoculated with bFGF in matrigel. B) Panels a and b: microscopic appearance of the sites of injection in a representative mouse treated with saline and inoculate with matrigel alone (panel a: 100× magnification; panel b: 100× magnification); panels c and d: microscopic appearance of the sites of injection in a representative mouse treated with saline and inoculate with bFGF in matrigel (panel c: 100× magnification; panel d: 100× magnification); panels e and f: microscopic appearance of the sites of injection in a representative mouse treated with indinavir and inoculate with bFGF in matrigel (panel e: 100× magnification; panel f: 100× magnification); panels g and h: microscopic appearance of the sites of injection in a representative mouse treated with saquinavir and inoculate with bFGF in matrigel (panel g: 100× magnification; panel h: 100× magnification).

FIG. 13 (panels A and B). Indinavir and saquinavir inhibit the invasive capacity of KS cells. Panel A: effect of indinavir on cell invasion; Panel B: effect of saquinavir on cell invasion.

FIG. 14. Indinavir and saquinavir do not interfere with the proliferation of endothelial/lung carcinoma hybrid (Ea-hy 926) cells.

FIG. 15. Indinavir and saquinavir inhibit the invasive capacity of endothelial/lung carcinoma hybrid (Ea-hy 926) cells.

FIG. 16. Indinavir and saquinavir do not interfere with the proliferation of hepato-carcinoma (SK-Hep-1) cells.

FIG. 17. Indinavir and saquinavir inhibit the invasive capacity of hepato-carcinoma (SK-Hep1) cells.

FIG. 18. Indinavir and saquinavir do not interfere with the proliferation of lung carcinoma (A549) cells.

FIG. 19. Indinavir and saquinavir inhibit the invasive capacity of lung carcinoma (A549) cells.

FIG. 20. Indinavir and saquinavir do not interfere with the proliferation of breast carcinoma (MDA-MB468) cells

FIG. 21. Indinavir and saquinavir Inhibit the invasive capacity of breast carcinoma (MDA-MB468) cells

FIG. 22. Indinavir and saquinavir inhibit the invasive capacity of myelo-monocytic leukaemia (U937) cells.

FIG. 23 (panels a, b, c, d, e and f). Indinavir and saquinavir inhibit the development of KS-like lesions induced by the inoculation of KS cells in the nude mice. Panels a and b: microscopic appearance of the site of KS cells injection in a representative mouse treated with saline solution (panel a: 100× magnification; panel b: 400× magnification); Panels c and d: microscopic appearance of the site of KS cells injection in a representative mouse treated with indinavir (panel c: 250× magnification; panel d: 400× magnification); Panels e and f: microscopic appearance of the site of KS cells injection in a representative mouse treated with saquinavir (panel e: 250× magnification; panel f: 400× magnification)

FIG. 24. Indinavir and saquinavir promote the regression of KS-like lesions induced by the inoculation of KS cells in nude mice.

FIG. 25. Indinavir and saquinavir promote the regression of tumour angiogenic lesions Induced by the inoculation of endothelial/lung carcinoma hybrid (Ea-hy 926) cells in nude mice.

FIG. 26. Indinavir and saquinavir inhibit the development of tumour lesions induced by the inoculation of hepatocarcinoma (SK-Hep-1) cells in nude mice.

FIG. 27. Indinavir and saquinavir inhibit the development of KS-like lesions induced by the inoculation of lung carcinoma (A549) cells in nude mice.

FIG. 28. Indinavir and saquinavir inhibit the development of tumour lesions induced by the inoculation of breast carcinoma (MDA-MB468) cells in nude mice.

FIG. 29. Indinavir and saquinavir inhibit the development of tumour lesions induced by the inoculation of myelo-monocytic leukaemia (U937) cells in nude mice.

FIG. 30. Indinavir and saquinavir inhibit the development of tumour lesions induced by the inoculation of T cell leukaemia (Jurkat) cells in nude mice.

FIG. 31. Indinavir and saquinavir block the vascular permeability and the oedema promoted by the inoculation of KS cells in the nude mouse.

FIG. 32 (panels A and B). Indinavir and saquinavir block the production of inflammatory cytokines such as IL-6 by KS cells. Panel A: Effect of indinavir on cytokine production; Panel B: effect of saquinavir on cytokine production.

### DETAILED DESCRIPTION OF THE INVENTION

Prior to describing the body of the invention, the following definitions are provided:

Cell invasion (invasion of cells)—A process by which cells migrate within a tissue or a basement membrane owing to the degradation of extracellular matrix or basement membranes by proteolytic enzymes

Tissue infiltration—the localisation of cells in a tissue upon their migration from distant sites owing to cell invasion

Oedema—The leakage of fluids from blood or lymphatic vessles due to the activity of infiltrating or resident cells releasing factors that alter the permeability of capillary endothelium and basement membrane structure due to the action of proteases, including matrix metalloproteases.

Extracellular matrix—a material produced by cells and filling spaces between cells and present in variable amounts in all tissues

Basement membrane—A proteinaceous structure produced by cells localised under normal endothelia or epithelia and separating them from underlying tissues.

Matrix metalloproteases—endopeptidases that can cleave virtually any component of the extracellular matrix that are divided in collagenases, gelatinases, stromelysins and matrilysins

Recent reports have described a reduced incidence or the regression of KS in HIV-1-infected patients treated with the highly active antiretroviral therapy (HAART) that contains at least one HIV-PI such as indinavir or saquinavir (Lebbé et al., 1998; Cattelan et al., 1999; International Collaboration on HIV and Cancer, 2000). These effects have been attributed by others to the blocking, by HIV-PI, of the replication of the HIV virus, to the blocking of the replication of the HHV8 virus and/or to the reconstitution of effective immune responses against HHV-8 and HIV (Blum et al., 1997; Rizzieri et al., 1997; Lebbé et al., 1998; De Milito et al., 1.999; Cattelan et al., 1999; Osman et al., 1999; Sirianni et al., 1999; Sirianni et al., 2000; Wang et al., 2000). On the other hand, our past and recent studies indicate that cytokines, growth and angiogenic factors (particularly bFGF) produced by KS cells, endothelial cells and cells of the immune system mediate the formation of KS lesions, and that endothelial and KS cell invasion, infiltration of tissues by these cells and inflammatory cells and immune cells, oedema formation, and activation or increased production of MMPs are key for KS lesion development and growth (Ensoli et al., 1989; Ensoli et al., 1994a; Fiorelli et al., 1995; Samaniego et al., 1995; Samaniego et al., 1997; Ensoli and Stürzl, 1998; Fiorelli et al., 1998; Samaniego et al., 1998; Barillari et al., 1999a; Barillari et al., 1999b; Fiorelli et al., 1999). So, contrary to the opinion commonly held in the scientific world, we hypothesised that the regression on KS in subjects with AIDS-KS treated with HIV-PI was due to a direct activity of HIV-PI on cell invasion, tissue infiltration and oedema due to effects on molecules and enzymes that are involved in these processes and are different from the cell proteasome. Using in vitro models, we showed that indinavir and saquinavir, used in the same concentrations present in plasma from treated patients, block primary macrovascular or microvascular endothelial cell and lymphoid or solid tumour cells migration and invasion, with no effects on the growth of these cells. Further, we showed that these HIV-PI inhibit the activation of an enzyme called MMP-2 or increased production of a casein-degrading MMP. The enzymes of the metalloprotease class (MMPS) are essential for cell motility (migration and invasion) or vascular permeability and, therefore, for the angiogenesis, oedema, and growth and invasion of tumours (Carmeliet and Jain, 2000). In agreement with these data, we demonstrated that indinavir and saquinavir block the development of KS-like angioproliferative lesions induced by the inoculation of bFGF, bFGF and VEGF combined or primary human KS cells in nude mice, and angiogenesis induced by bFGF or VEGF in the chicken chorioallantoic membrane (CAM). Further, we demonstrated that indinavir or saquinavir block the growth of tumours induced in nude mice by inoculation of human lymphoid and solid tumour cells. Finally, we showed that HIV-PI block vascular permeability and oedema promoted by the KS cells in nude mice. In addition, they inhibited production of cytokines by KS cells.

These cytokines not only promote KS lesions but have also inflammatory activity (Ensoli et al., 1989; Barillari et al., 1992; Samaniego et al., 1995; Fiorelli et al., 1995; Samaniego et al., 1997; Sirianni et al., 1998; Samaniego et al., 1998; Fiorelli et al., 1998; Fiorelli et al., 1999; Barillari et al., 1999a), and some of them also promote multicentric Castleman disease (MCD) and lymphomas (Tosato et al., 1993; Peterson and Frizzera, 1993; Ramsay et al., 1994; Asou et al., 1998).

These data, therefore, indicate that the effect of HIV-PI on KS and on lymphomas is due to a direct block of MMPs, migration and invasion of endothelial and tumour cells, with inhibitory effects for angiogenesis and oedema, determining the inhibition of the formation of lesions and the lower incidence of tumours observed in the murine model and/or in subjects treated with HIV-PI. These effects of HIV-PI, however, are not due to the inhibition of normal or neoplastic cell proliferation. It is important to stress that these therapeutic effects were obtained in the absence of HIV and HHV8, therefore excluding that these effects of HIV-PI could be mediated by these effects of HIV-PI on HIV and/or HHV8.

Thus, our studies indicate that HIV-PI can be exploited to modulate relevant biological processes or for the therapy of pathological conditions involving cell migration and invasion, tissue infiltration and activity of MMPs. In particular, the discovery that the inhibitors of HIV protease are powerful drugs at blocking cell invasion and tissue infiltration, and that they block the activity of cellular metalloproteases involved in these processes, opens a completely new field for modulation and treatment of all biological processes and pathological conditions related to the above cell responses and functions including angiogenesis, non-neoplastic angioproliferative pathologies, KS, tumours, inflammatory and autoimmune diseases, both in HIV-infected subjects and in non-HIV-infected subjects.

All those compounds that present activity as inhibitors of the protease of the HIV virus (referred here for brevity's sake as HIV-PI) and similar to or derived from the same therefore fall within the field of the present invention. In this regard, indinavir, saquinavir, ritonavir, nelfinavir, amprenavir, lopinavir are here mentioned as examples of these compounds.

HIV-PI compounds may be used as follows in both HIV-infected and non-HIV-Infected subjects:
for blocking the migration of endothelial cells with a therapeutic anti-angiogenic, anti-KS and anti-tumour effect
for blocking the migration of tumour cells with a therapeutic anti-KS and anti-tumour effect
for blocking the invasion of endothelial cells with a therapeutic anti-angiogenic, anti-KS and anti-tumour effect
for blocking the invasion of tumour cells with a therapeutic anti-KS and anti-tumour effect
for blocking migration of inflammatory cells with a therapeutic anti-inflammatory, anti-autoimmune, anti-angiogenic, anti-KS and anti-tumour effect
for blocking migration of immune cells with a therapeutic anti-inflammatory and anti-autoimmune effect
for blocking infiltration of tissues by inflammatory cells with a therapeutic anti-inflammatory, anti-autoimmune, anti-angiogenic, anti-KS and anti-tumour effect
for blocking infiltration of tissues by immune cells with a therapeutic anti-inflammatory and anti-autoimmune effect
for blocking MMPs including MMP-2, stromelysins, matrilysin and other proteases or molecules involved in cell migration and invasion; blocking enzymes activating MMPs and other proteases or molecules involved in cell migration and invasion; blocking thrombospondin and other molecules involved in cell migration and invasion;
for blocking MMPs, including MMP-2, stromelysins, matrilysin and the other proteases or molecules involved in angiogenesis (Carmeliet, Nature 2000)
for blocking enzymes activating MMPs and the other proteases involved in angiogenesis.
for blocking thrombospondin and other molecules involved in angiogenesis.
for blocking MMPs including MMP-2, stromelysins, matrylisin and the other proteases or molecules involved in migration of inflammatory and immune cells and tissue infiltration
for blocking MMPs, including MMP-2, and other proteases or molecules involved in the growth and metastasis of tumours
for blocking the activity of bFGF with a therapeutic anti-angiogenic, anti-tumour, anti-KS effect
for blocking the activity of VEGF with a therapeutic anti-angiogenic, anti-tumour, anti-KS, anti-oedemigenic effect
for blocking the activity of associated bFGF and VEGF with a therapeutic anti-angiogenic, anti-tumour, anti-KS, anti-oedemigenic effect
for blocking the activity of Tat alone or in the presence of bFGF with a therapeutic anti-angiogenic, anti-tumour, anti-KS, anti-oedemigenic and anti-inflammatory effect
for blocking vascular permeability and oedema associated with angiogenesis
for blocking vascular permeability and oedema associated with tumours
for blocking vascular permeability and oedema associated with KS
for blocking vascular permeability and oedema associated with inflammation
for blocking the production of inflammatory cytokines with a therapeutic anti-inflammatory effect
for blocking the production of cytokines with a therapeutic anti-oedemigenic effect for blocking the production of cytokines with a therapeutic anti-angiogenic effect
for blocking the production of cytokines with a therapeutic anti-KS effect
for blocking the production of cytokines with a therapeutic anti-tumour effect
for the therapy of KS
for the therapy of angiogenesis
for the therapy of non-neoplastic angioproliferative diseases (eye, kidney, vascular system, skin), such as, for example, diabetic retinopathy, retrolental fibroplasia, trachoma, vascular glaucoma, psoriasis, immune and non-immune inflammation, atherosclerosis, keloids
for the therapy of benign and malignant tumours of the soft tissues, the cartilages, the bones and the blood
for the therapy of autoimmune diseases in general, in particular systemic lupus erythematosus, scleroderma, rheumatoid arthritis, psoriasis, thyroiditis, ulcerous rectocolitis and Crohn's disease, Goodpasture's syndrome, systemic vasculitis, Sjögren's syndrome, primitive biliary cirrhosis for the therapy of inflammatory diseases, in particular of chronic inflammation associated with allergies and with viral infective, bacterial or parasitic agents, including the Castleman's multicentric disease.

For the above-mentioned uses all those compounds which manifest activity that inhibits the protease of the HIV virus are generally indicated, while particularly indicated are the compounds called indinavir, saquinavir, ritonavir, nelfinavir, amprenavir, lopinavir and those similar to or derived from them, alone or in a combination with one another and/or in combination with other drugs.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g., by means of conventional mixing, dissolving, granulating, dragée-making, levigating, emulsifying, encapsulating, entrapping or lyophilizing processes. Proper formulation is dependent upon the route of administration chosen. The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The chemical analogues and/or derivatives and/or salts of the known HIV-PI mentioned in the present description, alone or in combination one another and/or in association with other drugs or adjuvants or carriers or excipients, are considered within the scope of the invention.

The HIV-PI according to the invention may be given by oral, intravenous, intramuscular, subcutaneous, intradermal, intraperitoneal, intrathecal, intrapleural, intrauterine, intravaginal, topic intrarectal, transmucosal, intralesional or percutaneous administration, for all the indications listed above. The doses and the means of administration depend on the type of affection to be treated. In particular, doses are considered that are lower, equal to or higher than those commonly used for the treatment of HIV-infected patients. For example these doses are, for indinavir (about): 600 mg/day, 1200 mg/day, 2400 mg/day or 4800 mg/day; and for saquinavir (about): 900 mg/day; 1800 mg/day, 3600 mg/day, 7200 mg/day.

The examples given below, which also refer to the figures and tables enclosed, may be used for verifying our hypothesis. We used indinavir and saquinavir, two HIV-PI associated with the regression of KS in treated patients (Lebbé et al., 1998; Cattelan et al., 1999) that have a similar structure but chemical substituents designed to optimise their action. The effects of both the HIV-PI were studied in models of angiogenesis promoted by bFGF and/or VEGF in vivo and in vitro, on the formation of KS-like lesions and on vascular permeability induced in vivo by KS cells and on KS cells in vitro (Ensoli et al., 1989; Ensoli et al., 1994a and 1994b; Samaniego et al., 1995; Fiorelli et al., 1995; Samaniego et al., 1997; Samaniego et al., 1998; Barillari et al., 1999a; Sgadari et al., 2000), on the growth of tumours induced by lymphoid or solid tumour cell lines in vivo and on lymphold or solid tumour cell lines in vitro.

The following examples and figures are given to illustrate the invention and are not to be considered limiting the scope of the same.

Materials and Methods/Detailed Description of the Figures

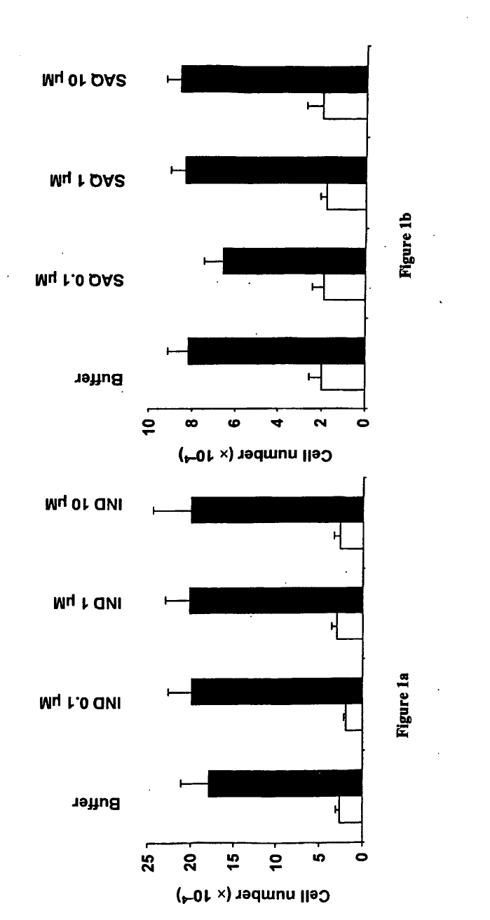

FIG. 1. Indinavir and saquinavir have no effect on the basal or bFGF-induced proliferation of primary macrovascular (humbelical vein) endothelial cells. Panel A: effect of indiinavir on the basal or bFGF-induced proliferation of primary macrovascular (humbelical vein) endothelial cells; Panel B: effect of saquinavir on the basal or bFGF-induced proliferation of primary macrovascular (humbelical vein) endothelial cells.

The figure shows the results of the proliferation assay expressed as the number of cells counted after 5 days of incubation with bFGF in PBS buffer (black bars) or without bFGF (PBS alone, white bars) in the presence or absence of 0.1, 1 or 10 μM of indinavir (IND) or saquinavir (SAQ) or their resupsension buffer (Buffer). Human endothelial cells from the umbilical vein (HUVEC, Blo-Whittaker, Verviers, Belgium) were plated in triplicate (1.5×10⁴ cells/well) in plates of 12 wells previously covered with gelatine. The next day the cells were incubated for 4 hours in a medium without serum and cultivated in RPMI 1640 medium (Life Technologies, Eragny, France) with the addition of 10% of foetal bovine serum (FBS) together with bFGF in PBS buffer (10 ng/ml) or PBS buffer alone. Medium, bFGF, PBS buffer, indinavir, saquinavir or HIV-PI resuspension buffer were replaced after 3 days. After 5 days' culture, the cells were counted after having been stained with trypan blue, as previously described (Ensoli et al., 1990; Ensoli et al., 1994b): For all the studies in vitro, the indinavir or the saquinavir in the pure powder formula (Merck Sharpe & Dohme and Roche, respectively) were re-suspended in distilled water. The drugs were found to be free from endotoxins on LAL testing (Associated of Cape Code Inc., Falmouth, Mass.).

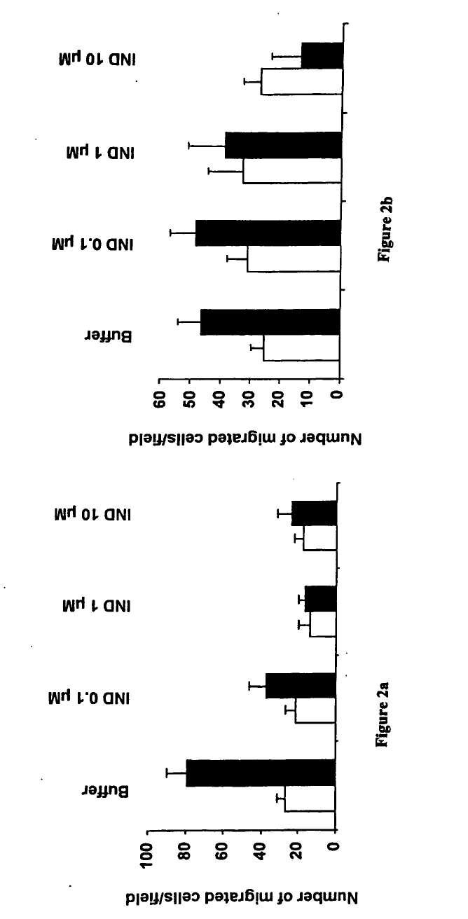

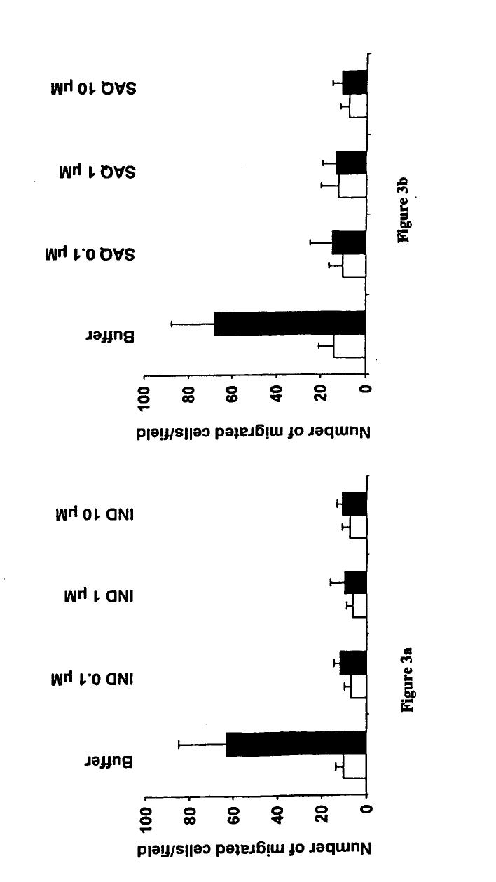

FIGS. 2 and 3. Indinavir and saquinavir block the migration and the invasion of primary macrovascular (humbelical vein) endothelial cells induced by bFGF.

FIG. 2 shows the results of the migration assay. FIG. 3 shows the results of the invasion assay. Both assays were performed on endothelial cells. Results are expressed as the number of cells/well that migrated (FIG. 2) or invaded (FIG. 3) in response to bFGF in PBS buffer (black bars) or in response to PBS buffer alone (white bars) in the presence 0.1, 1 or 10 μM of indinavir (IND), saquinavir (SAQ), or their dilution buffer (Buffer). Panels A: effect of indinavir on migration (FIG. 2) or invasion (FIG. 3) of primary macrovascular (humbelical vein) endothelial cells induced by bFGF; Panels B: effect of saquinavir on migration (FIG. 2) or invasion (FIG. 3) of primary macrovascular (humbelical vein) endothelial cells induced by bFGF.

Both assays were performed by the Boyden chamber separated in two compartments by polycarbonate filters with 12 μm pores (Nucleoprobe, Cabin John, Md.), coated with collagen IV (Collaborative Biomedical Products) for migration, or with collagen IV and Matrigel together for invasion, as described previously (Barillari et al., 1999b). The HUVEC were cultivated for 5-6 days in the presence of scalar concentrations of indinavir or saquinavir, or their dilution buffer. The cells were collected, re-suspended in a medium without serum containing 0.01% of BSA and placed in the upper compartment of the Boyden chamber in duplicate (2×10⁵ cells/well) in the presence of indinavir, saquinavir or their dilution buffer. bFGF (50 ng/ml) was placed in the lower compartment as a chemo-attractant in a medium containing 0.01% BSA. After 5 hours (migration) or 6 hours (Invasion) of incubation, the non-migrated cells present on the top surface of the filters were mechanically removed, while the migrated cells on the bottom surface were fixed in methanol and stained with toluidine blue (Sigma Chemical Co., St. Louis, Mo.). The cells present in 5-10 microscopic fields of the filters, chosen at random, were counted as described previously (Barillari et al., 1999b).

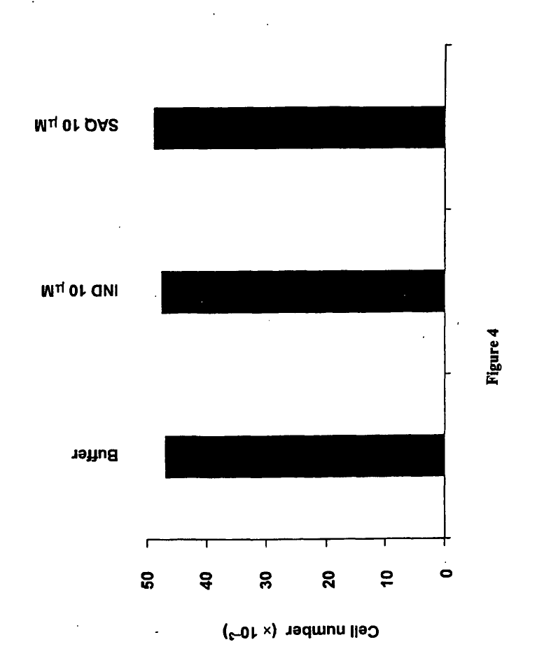

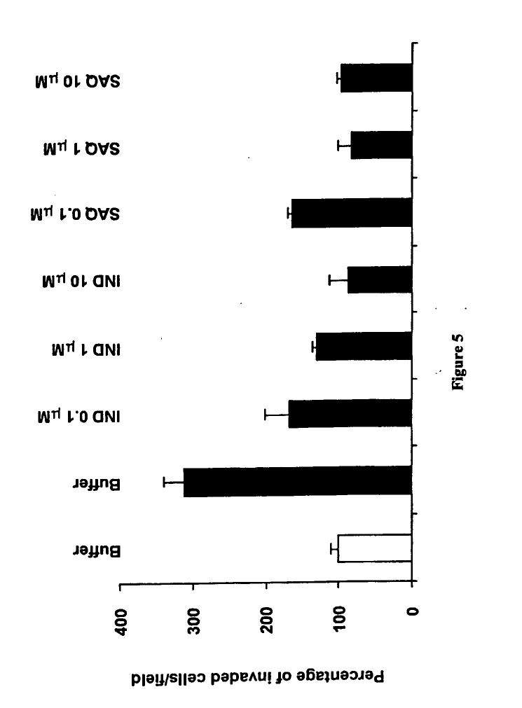

FIG. 4. Indinavir and saquinavir do not interfere with the proliferation of microvascular (dermal) endothelial cells in response to bFGF.

The figure shows the results of the proliferation assay expressed as the number of dermal microvascular endothelial cells counted after 5 days of incubation with bFGF in the presence of 10 μM of indinavir (IND) or saquinavir (SAQ) or their resuspension buffer (Buffer). Human dermal microvascular endothelial cells (H-DMVEC, Bio-Whittaker) were seeded in triplicate wells (2×10⁴ cells/well) in gelatin-coated 12-well plates and cultured in RPMI 1640 medium supplemented with 10% FBS in the continuos presence of bFGF (10 ng/ml) to prevent apoptosis occurring after 24-48 hours upon depletion of this factor. Media, bFGF, indinavir, saquinavir (10 μM) or HIV-PI resuspension buffer were replaced after 3 days. After 5 days of culture, cells were counted by trypan blue dye exclusion, as described above. Data from duplicate experiments are expressed as the number of H-DMVEC grown in response to bFGF in the presence of indinavir, saquinavir or HIV-PI-resuspension buffer.

FIG. 5. Indinavir and saquinavir inhibit the invasion of microvascular (dermal) endothelial cells in response to bFG F.

The figure shows the results of cell invasion assays performed with H-DMVEC. Data are expressed as the mean percentage and standard deviations (SD) of cells invaded in response to bFGF in PBS buffer (▪) or PBS buffer alone (□) in the presence of indinavir (IND), saquinavir (SAQ) or HIV-PI-resuspension buffer (Buffer). Basal invasion in the absence of bFGF was assumed as 100%. Data of triplicate experiments by the Boyden chamber assay performed as described above for HUVEC are shown. The block of H-MVDEC invasion resulted statistically significant at 10 mM for indinavir and at 1 and 10 mM for saquinavir, P<0.05).

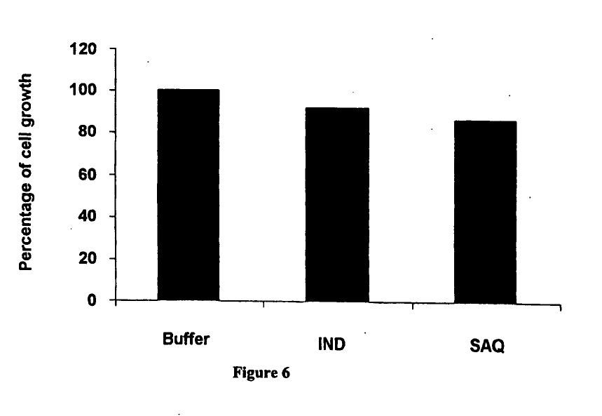

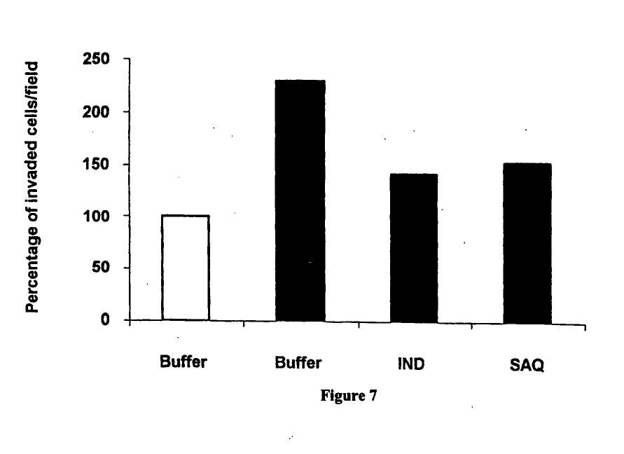

FIGS. 6 and 7. Indinavir and saquinavir inhibit the invasion but not the proliferation of smooth muscle cells in response to bFGF.

FIG. 6 shows the results of the cell growth and FIG. 7 of cell invasion assays performed with smooth muscle cells. Assays have been essentially performed as described in key to FIGS. 1 and 3. Data are expressed as the number or the percentage of cells grown or invaded in response to bFGF in PBS buffer (▪) or PBS buffer alone (□) in the presence of indinavir (IND), saquinavir (SAQ) or HIV-PI-resuspension buffer (Buffer), as indicated. bFGF-induced cell growth in the absence of HIV-PI or basal cell invasion in the absence of bFGF were assumed as 100%. Data of duplicate experiments (mean) are shown.

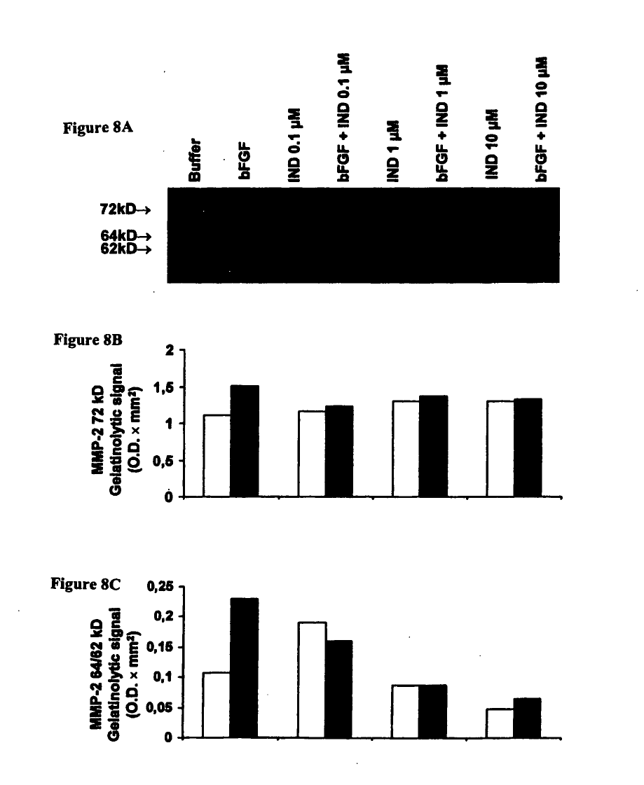

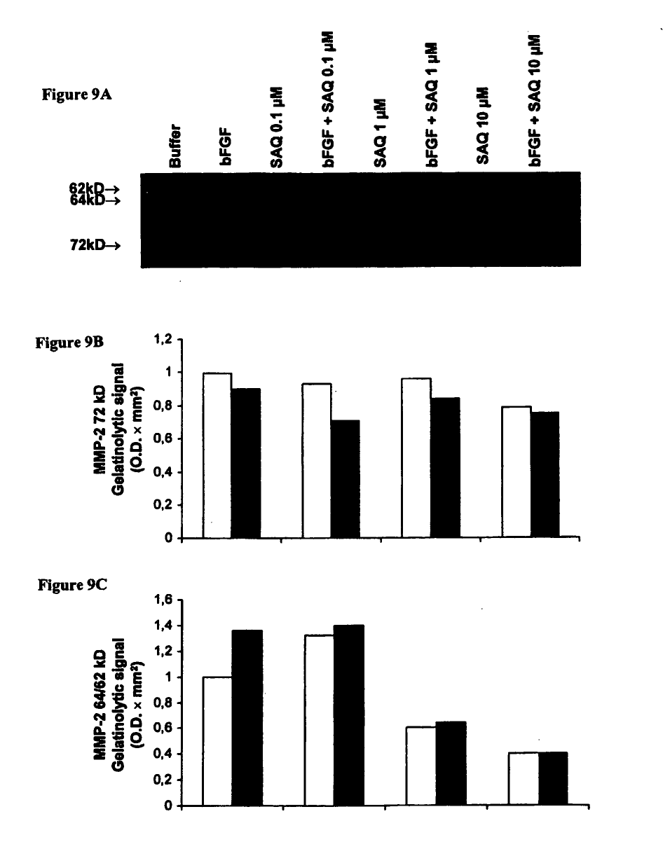

FIGS. 8 and 9. Indinavir and saquinavir block the conversion of latent MMP-2 into its active form. FIG. 8: effect of indinavir on MMP-2 activation. Panel A: zymography assay carried out on concentrated supernatants from HUVEC stimulated with bFGF in PBS (black bars) or PBS buffer alone (Buffer, white bars), and cultivated for 24 hours in the presence of 0.1, 1 or 10 μM indinavir (IND) or their resuspension buffer. The arrows indicate the de-stained areas due to gelatinolytic activity corresponding to the latent form (72 kD), pre-activated form (64 kD) and active form (62 kD) of MMP-2. Panel B: densitometric quantification of the de-stained areas corresponding to the gelatinolytic activity of the latent form 72 kD; Panels C: densitometric quantification of the de-stained areas corresponding to the gelatinolytic activity of the pre-MMP-2 (64 kD) and active form (kD 62) of MMP-2 released by the cells. The results are expressed as the optical density of the de-stained bands. FIG. 9: effect of saquinavir on MMP-2 activation. Panel A: zymography assay carried out on concentrated supernatants from HUVEC stimulated with bFGF in PBS (black bars) or PBS buffer alone (Buffer, white bars), and cultivated for 24 hours in the presence of 0.1, 1 or 10 μM saquinavir (SAQ) or their resuspension buffer. The arrows indicate the de-stained areas due to gelatinolytic activity corresponding to the latent form (72 kD), pre-activated form (64 kD) and active form (62 kD) of MMP-2. Panel B: densitometric quantification of the de-stained areas corresponding to the gelatinolytic activity of the latent form 72 kD; Panels C: densitometric quantification of the de-stained areas corresponding to the gelatinolytic activity of the pre-MMP-2 (64 kD) and active form (kD 62) of MMP-2 released by the cells. The results are expressed as the optical density of the de-stained bands.

HUVEC were cultivated for 24 hours in RPMI 1640 with the addition of 10% FBS in the presence of scalar concentrations of indinavir, saquinavir or the diluting buffer, in the absence or presence of bFGF (100 ng/ml). The cells were then washed twice with a medium without serum and incubated all night in a medium without serum in the presence of the same concentrations of HIV-PI. The supernatants of the cellular cultures were then collected and concentrated using Centricon-10 (Amicon, Bedford, Mass.). The protein concentration was determined by means of Bradford analysis (Bio-Rad, Hercules, Calif.) using the BSA as standard. Equal quantities (5 μg) of protein were then diluted in a buffer for zymography (5×) (0.4 M Tris-HCl, pH 6.8, 5% SDS, 20% glycerol and 0.03% bromphenol blue) and loaded on polyacrylamide gel with 9% of SDS containing 1 mg/ml of gelatine. After electrophoresis, the gels were incubated for 1 hour in 2.5% (v/v) of Triton X-100 to eliminate the SDS and subsequently with an enzymatic buffer (50 mM Tris-HCl, pH 7.5, 200 mM NaCl, 5 mM CaCl2, 0.02% Brij-35) for the whole night at 37° C., as described previously (Kleiner et al., 1993). The gels were then stained with 2.5% Comassie blue G-250 and de-stained in 30% methanol and 10% acetic acid. The densitometry of the de-stained areas was then quantified using a densitometer GS-700 connected to a Macintosh Performa computer with Multi-Analyst software (Bio-Rad).

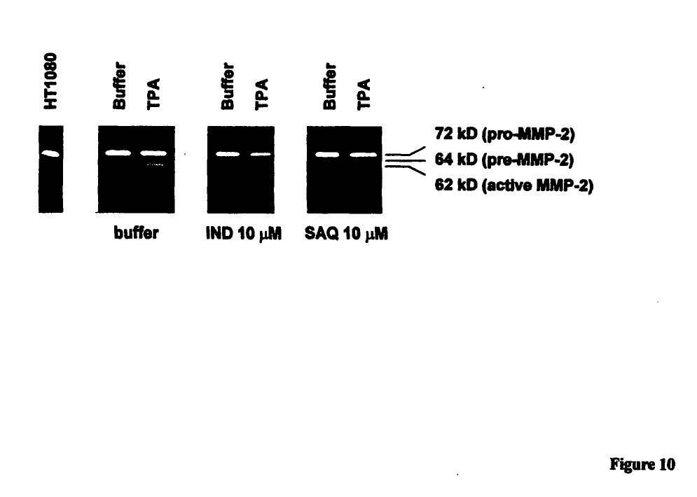

FIG. 10. Indinavir and saquinavir block the autoproteolytic conversion of pre-MMP-2 to its active form. The figure shows a zymography assay performed on supernatants from HUVEC stimulated with a phorbol ester (12-O-Tetradecanoylphorbol-13-acetate) (TPA) (50 nM) or its re-suspension buffer (Buffer), and cultivated for 8 hours in the presence of 10 μM of indinavir (IND), saquinavir (SAQ), or their resuspension buffer (Buffer). The arrows indicate the de-stained areas due to gelatinolytic activity corresponding to the latent form (pro-MMP2, 72 kD), pre-activated form (pre-MMP2, 64 kD) and active form (active MMP2, 62 kD) of MMP-2. HUVEC were cultivated for 24 hours in RPMI 1640 with the addition of 10% FBS. Before assay, cells were washed twice with a medium without serum and incubated over night in a medium without serum containing 0.01% w/v bovine serum albumin (BSA) in the presence of 10 mM indinavir or saquinavir or their resuspension buffer. Cells were then incubated for 8 hours in medium containing 0.01% w/v BSA, 10 mM indinavir or saquinavir, and TPA, or its resuspension buffer. Aliquots of cell supernatants were then assayed for MMP-2 activity by gel zymography as described above. HT1080 is a tumour cell line secreting large amount of MMP-2 that was used as a control. As shown in the Figure, treatment of HUVEC with TPA induced the conversion of pro-MMP2 to pre- and active MMP2. However, in the presence of indinavir or saquinavir the relative intensity of the active MMP2 gelatinolytic band was decreased as compared to pre-MMP2, indicating a block of the autoproteolytic activation of MMP-2 in its active form by both indinavir and saquinavir. Indinavir also inhibited the total amount of MMP-2 released by cells in the presence of TPA. The amount of supernatant analysed was normalised to the total cell number.

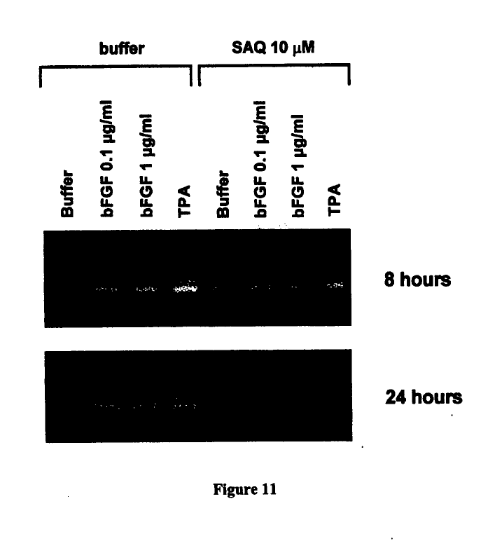

FIG. 11. Saquinavir blocks the production of casein-degrading MMPs by endothelial cells. HUVEC were grown and treated as described in FIG. 10, except that after a over night incubation in the absence of serum, cells were cultured in the presence of TPA (50 nM), different concentrations of bFGF (0.1 Or 1 μg/ml) in PBS buffer, PBS buffer alone (Buffer) in the presence of saquinavir (SAQ) or its resuspension buffer (buffer). Aliquots of cell supernatants normalised to total cell number were then analysed for MMP activity by gel zymography as described above in gels containing casein (2 mg/ml) instead of gelatin.). Panel A: casein zymography performed with cell supernatants harvested after 8 hours of culture in the presence of TPA or bFGF; Panel B: casein zymography performed with cell supernatants harvested after 24 hours of culture in the presence of TPA or bFGF. Casein is specifically cleaved by stromelysins (MMP-3, MMP-10, MMP-11) and matrilysin (MMP-7) (Whittaker and Ayscough, 2001) This led to the appearance of destained areas due to caseinolytic activity present in cell supernatants. As shown in the figure, in the absence of saquinavir, bFGF induced in a dose-dependent fashion the release of casein-degradation activity, that was maximal for cells treated with TPA. However, saquinavir (10 μM) inhibited the release of this activity by both bFGF or TPA, as indicated by the lower intensity of caseinolytic bands.

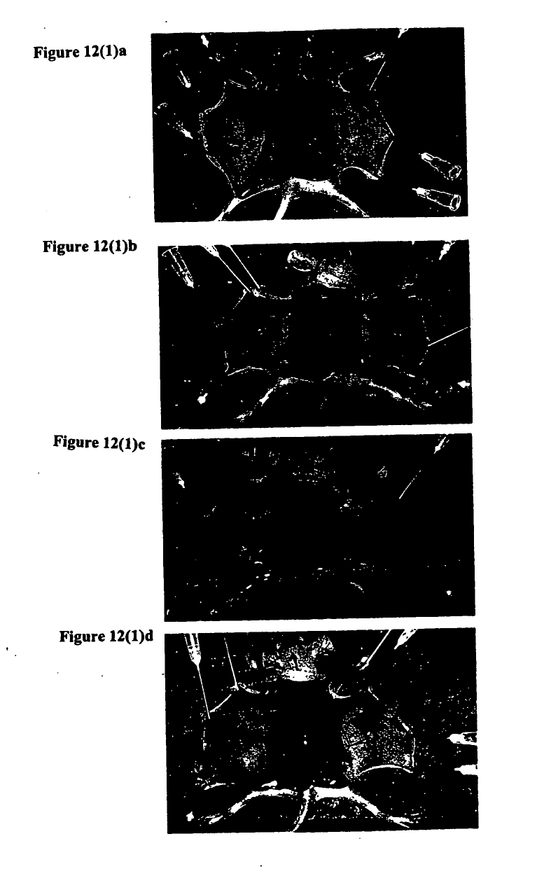

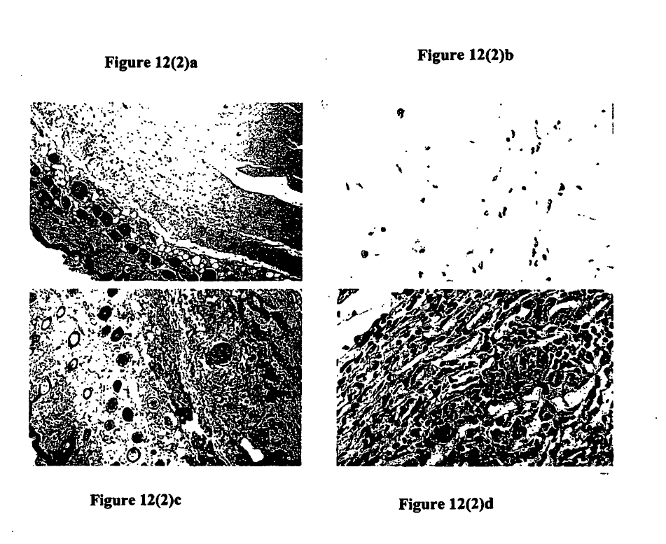

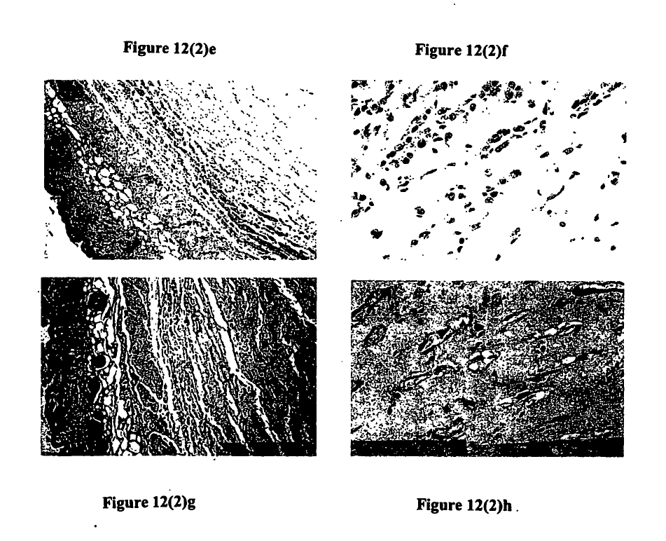

FIG. 12. Indinavir and saquinavir block the formation of KS-like angiogenic lesions induced by bFGF in nude mice.

(A) panel a: macroscopic appearance of sites of injection present in mice injected on the two sides with a buffer (PBS-0.1% BSA) in matrigel and treated with saline solution; panel b: macroscopic appearance of sites of injection present in mice injected on the two sides with bFGF (1 μg) in matrigel and treated with saline solution; panel c: macroscopic appearance of sites of injection present in mice injected with bFGF (1 μg) in matrigel and treated with indinavir (1.4 mg/day); panel d: macroscopic appearance of sites of injection present in mice injected with bFGF (1 μg) in matrigel and treated with saquinavir (1 mg/day). (B) panel a: microscopic appearance of the site of inoculation stained with H&E of a representative mouse injected with buffer and treated with saline solution (100× magnification); panel b: microscopic appearance of the site of inoculation stained with H&E of a representative mouse injected with buffer and treated with saline solution (400× magnification); panel c: microscopic appearance of the site of inoculation stained with H&E of a representative mouse injected with bFGF and treated with saline solution (100× magnification); panel d: microscopic appearance of the site of inoculation stained with H&E of a representative mouse injected with bFGF and treated with saline solution (400× magnification); panel e: microscopic appearance of the site of inoculation stained with H&E of the site of inoculation of a representative mouse injected with bFGF and treated with indinavir (100× magnification); panel f: microscopic appearance of the site of inoculation stained with H&E of the site of inoculation of a representative mouse injected with bFGF and treated with indinavir (400× magnification); panel g: microscopic appearance of the site of inoculation stained with H&E of the site of inoculation of a representative mouse injected with bFGF and treated with saquinavir (100× magnification); panel h: microscopic appearance of the site of inoculation stained with H&E of the site of inoculation of a representative mouse injected with bFGF and treated with saquinavir (400× magnification). The experiments were carried out as described in the key to Table 1.

FIG. 13. Indinavir and saquinavir inhibit the invasive capacity of KS cells. Panel A: effects of indinavir on invasion of two KS cells strains (KS3, KS8); Panel B: effects of saqinavir on invasion of two KS cells strains (KS8, KS12).

The figure shows the result of invasion assays carried out on three different primary KS cell strains (KS3, KS8, KS12) cultivated in vitro for 5-6 days in the presence of indinavir or saquinavir or diluting buffer (control). Both drugs inhibited the capacity of the KS cells to invade the Matrigel membrane in a dose-dependent manner. In particular, both the HIV-PI inhibited invasion as compared to the levels observed in control KS cells (p<0.05).

The assay was performed as described in FIG. 3. Briefly, the KS cells were cultivated for 5-6 days in the presence of indinavir or saquinavir (1 μM) or of the diluting buffer (saline solution). The cells were harvested and then plated in duplicate (5×10⁵ in a culture medium containing 0.05% BSA) in the upper compartment of the Boyden chamber, always in the presence of HIV-PI or buffer. bFGF (20 ng/ml) was placed in the bottom compartment as a chemo-attractant.

After 6 hours the cells that invaded the matrigel membrane were stained and counted as described in the key to FIG. 3.

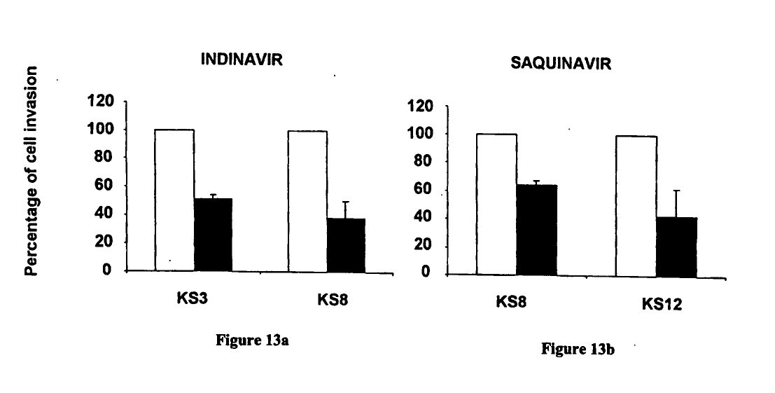

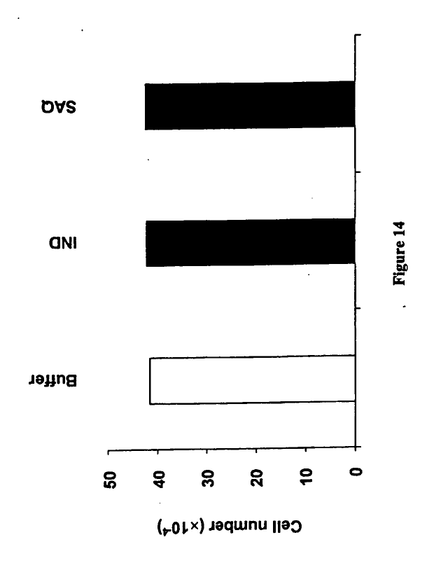

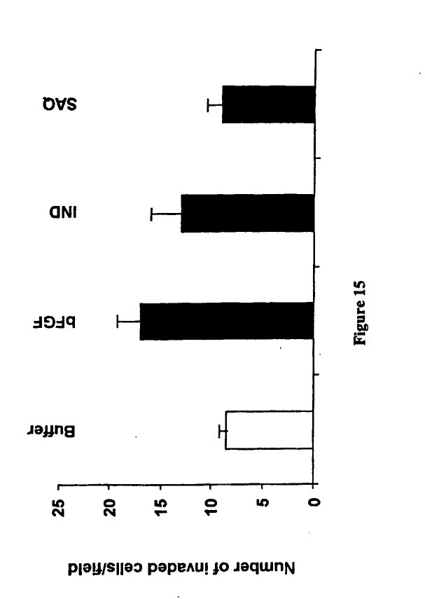

FIGS. 14 and 15. Indinavir and saquinavir inhibit the invasion but not the proliferation of endothelial/lung carcinoma hybrid (Ea-hy 926) cells.

FIG. 14 shows the results of cell growth assays performed with Ea-hy 926 cells, a hybrid between H-UVEC and human lung adeno-carcinoma cells (Edgell et al., 1983), which retains most of the endothelial cell markers and is used as an angiogenic tumour model (Albini et al., 1995, Albini et al., 1996, Cai et al, 1999). Results are expressed as the number of cells counted 5 days after the addition of 1 μM of indinavir (IND) or saquinavir (SAQ) (▪) as compared to the HIV-PI-resuspension buffer (□) (Buffer). The assays have been performed essentially as described in key to FIG. 1. No growth factors were added to EA-hy 926 cells since they produce factors which mediates cell growth in autocrine fashion (Edgell et al., 1983, Albini et al., PNAS 1995, Albini et al., Nat Med 1996). FIG. 15 shows the results of the invasion assays expressed as the number of invaded cells/field in response to bFGF in the presence of 1 μM of indinavir (IND) or saquinavir (SAQ) (▪) as compared to the HIV-PI-resuspension buffer (□) (Buffer). The assays have been performed essentially as described in legend to FIG. 3. Average and variation range of two independent experiments each performed in duplicate are shown. The block of EA-hy 926 cell invasion upon saquinavir treatment resulted statistically significant (P<0.05).

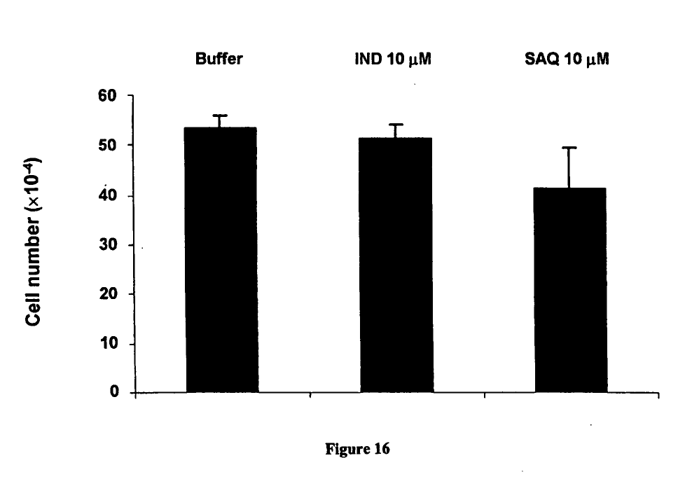

FIG. 16. Indinavir and saquinavir do not interfere with the proliferation of hepato-carcinoma cells (SK-Hep-1). Shown is the result of a representative cell growth assay performed on the hepato-carcinoma cells. Human hepato-carcinoma cells (SK-Hep-1; from ATCC) were plated in triplicate (8×10⁴ cells/well) in 12-well plates. The day after, following a 6 hours starvation in serum-free medium, the cells were incubated in culture medium containing 10% of foetal bovine serum (FBS) in the presence of 10 μM of indinavir (IND) or saquinavir (SAQ) or diluting buffer (PBS). The medium containing indinavir, saquinavir or the buffer was replaced every 2 days. After 4 days of culture, the cells were counted by trypan blue dye staining, as previously described (Ensoli et al., 1990; Ensoli et al., 1994b): For all the in vitro studies HIV-PI as endotoxin-free pure powder (a kind gift of Merck Sharp & Dohme and Roche) were resuspended in distilled water. Assays were repeated at least three times.

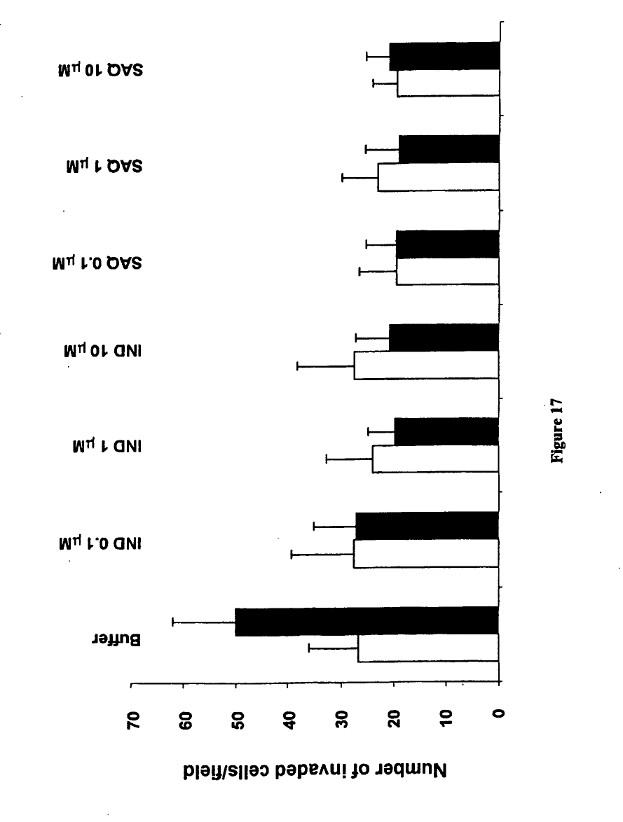

FIG. 17. Indinavir and saquinavir inhibit the invasive capacity of hepato-carcinoma (SK-Hep-1) cells in response to bFGF.

Shown are the averages of invading hepato-carcinoma cells SK-Hep-1 from two different experiments expressed as the mean number of invading cells in response to bFGF (black bars) or to its diluting buffer (white bars) in the presence of 0.1, 1 or 10 μM of indinavir (IND) or saquinavir (SAQ) or their dilution buffer (Buffer). The invasion assays were performed in the Boyden chamber. Polycarbonate filters (8 μM pores; Nucleoprobe, Cabin John, Md.) were coated first with collagen IV and then with Matrigel (Collaborative Biomedical Products) as described previously (Barillari et al., 1999b). The SK-Hep-1 cells were cultivated for 5 days in the presence of scalar concentrations of indinavir or saquinavir (0.1 μM, 1 μM, 10 μM), or the diluting buffer (control). 2×10⁵ cells were plated in duplicate in the upper compartment of Boyden chamber in 0.1% BSA containing increasing concentration of indinavir, saquinavir or the diluting buffer. Human recombinant bFGF (50 ng/ml) was placed in the lower compartment as chemo-attractant. After 5 hours of incubation, the non-invaded cells remaining on the upper surface of the filters were mechanically removed, whereas the cells invaded in the lower surface of the filters were fixed in ethanol and stained with toluidine blue (Sigma Chemical Co., St. Louis, Mo.). Ten random filter fields were counted by light microscopy as described previously (Barillari et al., 1999b).

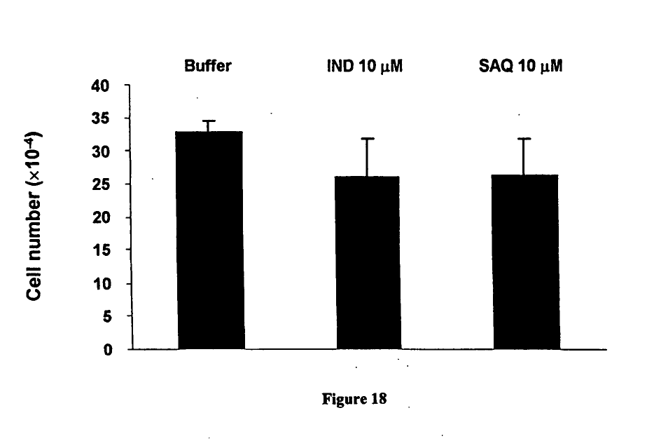

FIG. 18. Indinavir and saquinavir do not interfere with the proliferation of lung carcinoma cells (A549).

Shown is the result of a representative cell growth assay performed on the lung carcinoma cells. Human lung carcinoma cells (A549; ATCC) were plated in triplicate (8×10⁴ cells/well) in 12-well plates. The day after, following a 6 hours starvation in serum-free medium, the cells were incubated in culture medium containing 10% of foetal bovine serum (FBS) in the presence of 10 μM of indinavir (IND) or saquinavir (SAQ) or dilution buffer (Buffer). The medium containing indinavir, saquinavir or the buffer was replaced every 2 days. After 4 days of culture, the cells were counted by trypan blue dye staining, as previously described (Ensoli et al., 1990; Ensoli et al., 1994b): For all the in vitro studies HIV-PI as endotoxin-free pure powder (a kind gift of Merck Sharp & Dohme and Roche) were resuspended in distilled water. Assays were repeated at least three times.

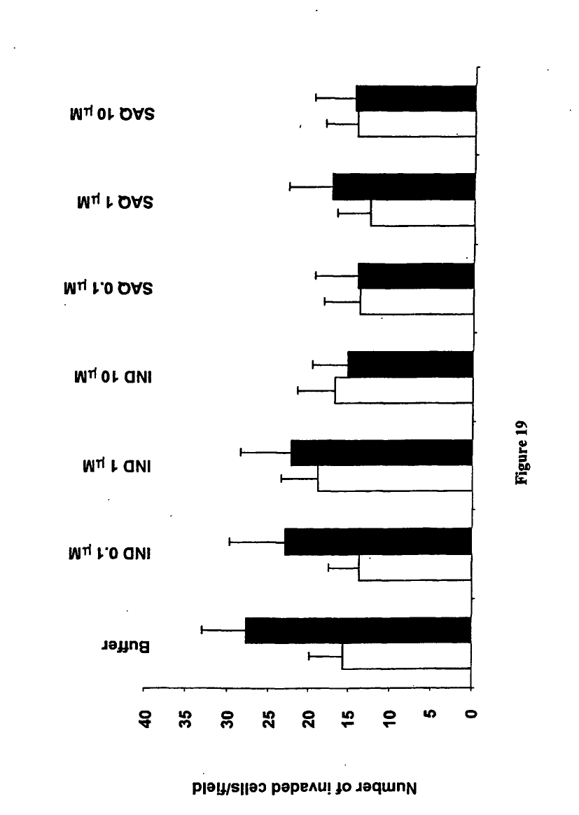

FIG. 19. Indinavir and saquinavir inhibit the invasive capacity of lung carcinoma (A549) cells in response to bFGF.

Shown are the averages of invading lung carcinoma cells A549 from two different experiments expressed as the mean number of invading cells in response to bFGF (black bars) or to its dilution buffer (white bars) in the presence of 0.1, 1 or 10 μM of indinavir (IND) or saquinavir (SAQ) or of the diluting buffer (Buffer); The invasion assays were performed in the Boyden chamber. Polycarbonate filters (12 μM pores; Nucleoprobe, Cabin John, Md.) were coated first with collagen IV and then with Matrigel (Collaborative Biomedical Products) as described previously (Barillari et al., 1999b). The A549 cells were cultivated for 5 days in the presence of scalar concentrations of indinavir or saquinavir (0.1 μM, 1 μM, 10 μM), or the diluting buffer. 2×10⁵ cells were plated in duplicate in the upper compartment of Boyden chamber in 0.1% BSA containing increasing concentration of indinavir, saquinavir or the diluting buffer. Human recombinant bFGF (50 ng/ml) was placed in the lower compartment as chemo-attractant. After 5 hours of incubation, the non-invaded cells remaining on the upper surface of the filters were mechanically removed, whereas the cells invaded in the lower surface of the filters were fixed in ethanol and stained with toluidine blue (Sigma Chemical Co., St. Louis, Mo.). Ten random filter fields were counted by light microscopy as described previously (Barillari et al., 1999b).

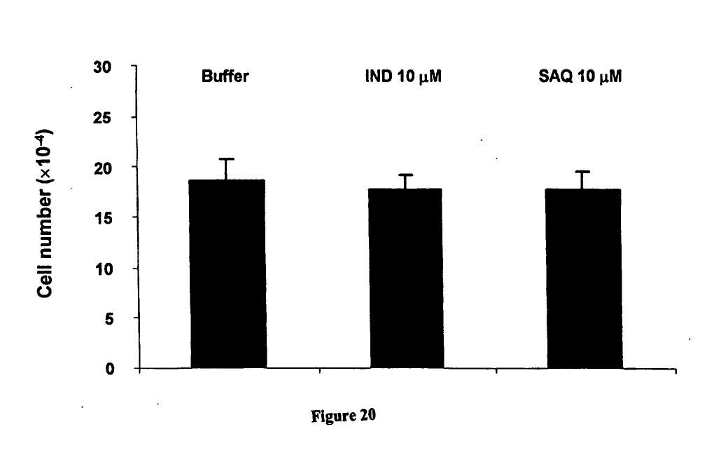

FIG. 20. Indinavir and saquinavir do not interfere with the proliferation of breast carcinoma cells (MDA-MB-468).

Shown is the result of a representative cell growth assay performed on the breast carcinoma cells. Human breast carcinoma cells (MDA-MB468; ATCC) were plated in triplicate (8×10⁴ cells/well) in 12-well plates. The day after, following a 6 hours starvation in serum-free medium, the cells were incubated in culture medium containing 10% of foetal bovine serum (FBS) in the presence of 10 μM of indinavir (IND) or saquinavir (SAQ) or diluting buffer (Buffer). The medium containing indinavir, saquinavir or the buffer was replaced every 2 days. After 4 days of culture, the cells were counted by trypan blue dye staining, as previously described (Ensoli et al., 1990; Ensoli et al., 1994b): For all the in vitro studies HIV-PI as endotoxin-free pure powder (a kind gift of Merck Sharp & Dohme and Roche) were resuspended in distilled water. Assays were repeated at least three times.

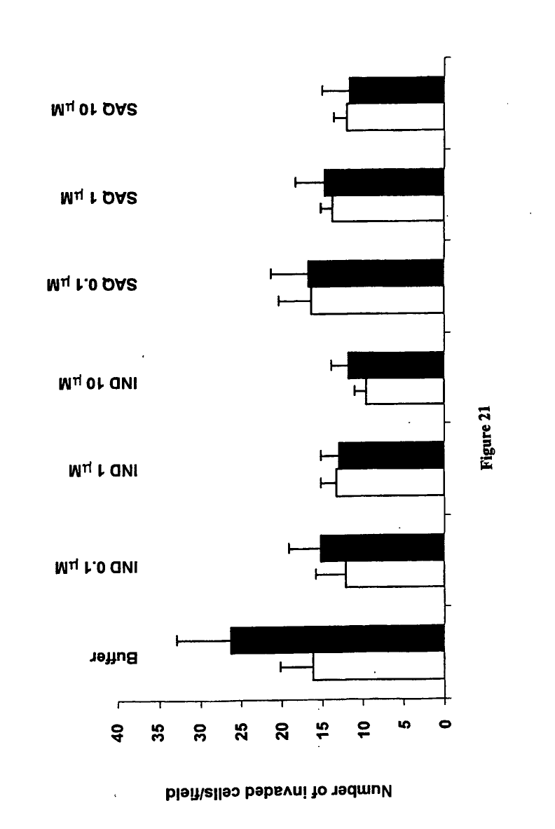

FIG. 21. Indinavir and saquinavir inhibit the invasive capacity of breast carcinoma (MDA-MB468) cells in response to bFGF.

Shown is the result of a representative invasion assay performed on the breast carcinoma cells MDA-MB468. Data are expressed as the mean number of invading cells in response to bFGF (black bars) or to its diluting buffer (white bars) in the presence of 0.1, 1 or 10 μM of indinavir (IND) or saquinavir (SAQ) or of their diluting buffer (Buffer). The invasion assays were performed in the Boyden chamber. Polycarbonate filters (8 μM pores; Nucleoprobe, Cabin John, Md.) were coated first with collagen IV and then with Matrigel (Collaborative Biomedical Products) as described previously (Barillari et al., 1999b). The breast carcinoma cells (MDA-MB468) were cultivated for 5 days in the presence of scalar concentrations of indinavir or saquinavir (0.1 μM, 1 μM, 10M), or the diluting buffer. 2×10⁵ cells were plated in duplicate in the upper compartment of Boyden chamber in 0.1% BSA containing increasing concentration of indinavir, saquinavir or the diluting buffer. Human recombinant bFGF (50 ng/ml) was placed in the lower compartment as chemoattractant. After 5 hours of incubation, the non-invaded cells remaining on the upper surface of the filters were mechanically removed, whereas the cells invaded in the lower surface of the filters were fixed in ethanol and stained with toluidine blue (Sigma Chemical Co., St. Louis, Mo.). Ten random filter fields were counted by light microscopy as described previously (Barillari et al., 1999b). Assays were repeated two times.

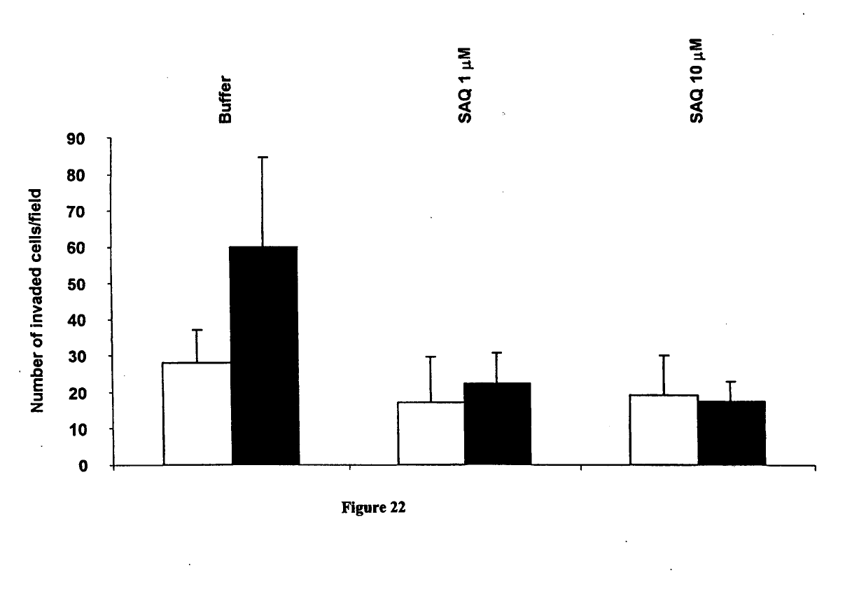

FIG. 22. Indinavir and saquinavir inhibit the invasive capacity of myelo-monocytic leukaemia (U937) cells in response to bFGF.

Shown is the result of a representative invasion assay performed on the myelo-monocytic leukaemia cells U937. Data are expressed as the mean number of invading cells in response to bFGF (black bars) or to its diluting buffer (white bars) in the presence of 1 or 10 μM of saquinavir or their diluting buffer (Buffer). Similar results were obtained also when the cells were treated with 1 or 10 μM of indinavir (data not shown). The invasion assays were performed in the Boyden chamber. Polycarbonate filters (5 μM pores; Nucleoprobe, Cabin John, Md.) were coated first with collagen IV and then with Matrigel (Collaborative Biomedical Products) as described previously (Barillari et al., 1999b). The myelo-monocytic leukaemia cells U937 were cultured for 4 days in the presence of scalar concentrations of indinavir or saquinavir (1 μM, 10 μM), or the diluting buffer. 8×10⁵ cells were plated in duplicate in the upper compartment of Boyden chamber in 0.1% BSA containing increasing concentration of indinavir, saquinavir or the diluting buffer. Human recombinant bFGF (50 ng/ml) was placed in the lower compartment as chemoattractant. After 4 hours of incubation, the non-invaded cells remaining on the upper surface of the filters were mechanically removed, whereas the cells invaded in the lower surface of the filters were fixed in ethanol and stained with toluidine blue (Sigma Chemical Co., St. Louis, Mo.). Ten random filter fields were counted by light microscopy as described previously (Barillari et al., 1999b). Assays were repeated two times.

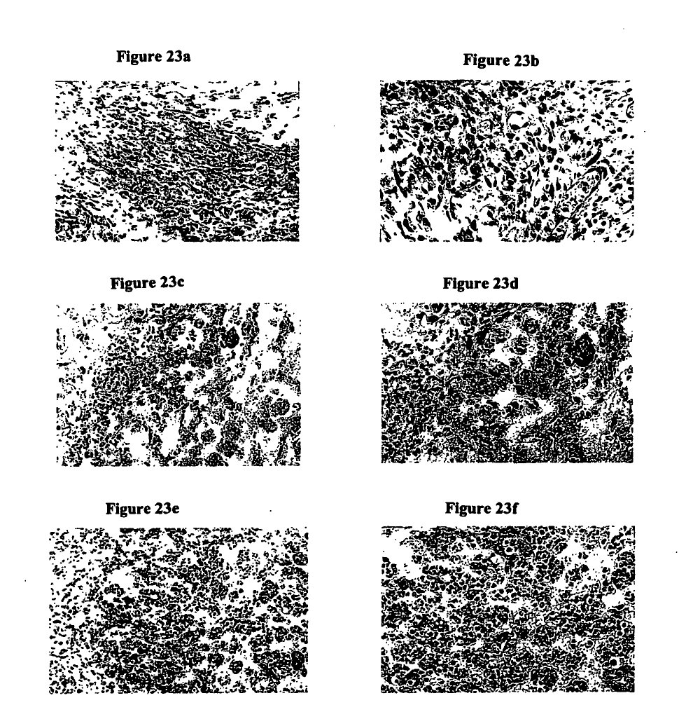

FIG. 23. Indinavir and saquinavir inhibit the development of KS-like lesions induced by the inoculation of KS cells in the nude mice.

The nude mice were inoculated with KS cells (3×10⁶) to induce the formation of angioproliferative KS-like lesions or with its re-suspension buffer (control) and treated with indinavir, saquinavir or saline solution according to the doses and procedures described in the key to FIG. 12, starting 2 days before cell inoculation. At the time of sacrifice, the sites of inoculation were examined to check for the presence of macroscopic angioproliferative lesions as described in the key to FIG. 12 and Table 5. Panel a: microscopic appearance of the central area of the site of KS cell inoculation stained with H&E in a representative mouse treated with saline solution (100× magnification); panel b: microscopic appearance of the central area of the site of KS cell inoculation stained with H&E in a representative mouse treated with saline solution (400× magnification); panel c: microscopic appearance of the central area of the site of KS cell inoculation stained with H&E in a representative mouse treated with indinavir (250× magnification); panel d: microscopic appearance of the central area of the site of KS cell inoculation stained with H&E in a representative mouse treated with indinavir (400× magnification); panel e: microscopic appearance of the central area of the site of KS cell inoculation stained with H&E in a representative mouse treated with saquinavir (250× magnification); panel f: microscopic appearance of the central area of the site of KS cell inoculation stained with H&E in a representative mouse treated with saquinavir (400× magnification); The experiments were carried out as described in the key to Table 5.

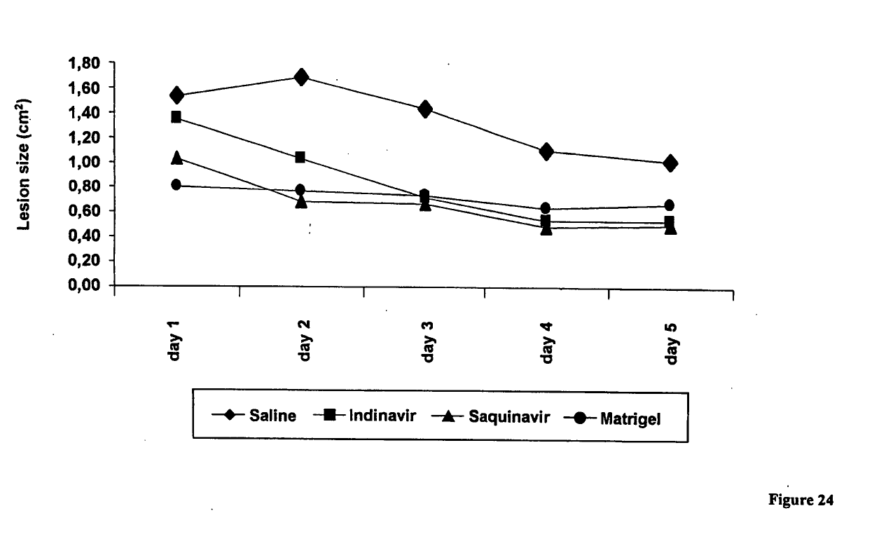

FIG. 24. Indinavir and saquinavir promote the regression of KS-like lesions induced by the inoculation of KS cells in nude mice.

Indinavir and saquinavir can also promote KS regression in the absence of any drug pre-treatment. Nude mice (10 animals/group) were inoculated with KS cells (KS12 cell strain, 3×10⁶) to induce the formation of angioproliferative KS-like lesions or with its re-suspension buffer (control) and on the same day started the treatment with indinavir, saquinavir or saline solution by intragastric gavage according to the doses described in the legend to Table 5. Treatment was then continued for 5 days, until sacrifice. The mean size (cm²) of the lesions present at the injection site evaluated by daily caliper measurement and calculated as the product of the two major lesion diameters is shown.

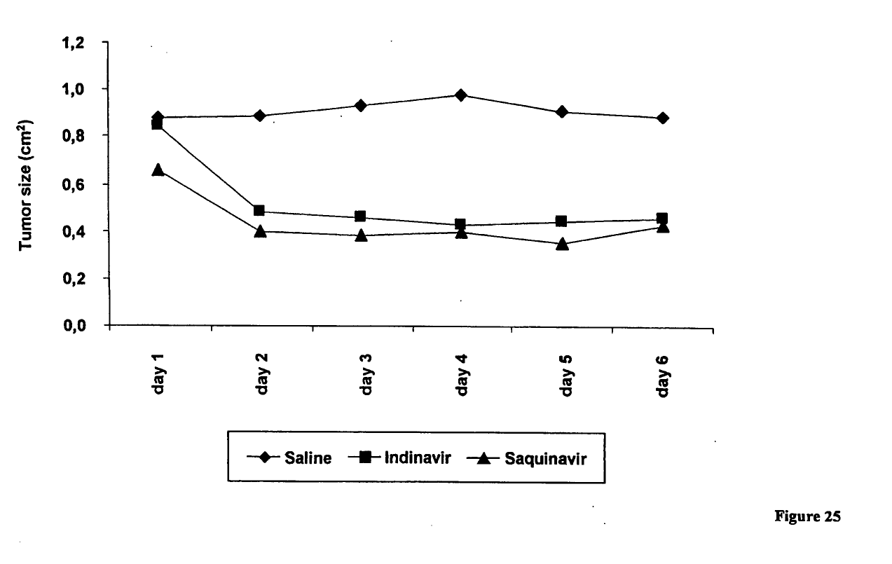

FIG. 25. Indinavir and saquinavir promote the regression of tumour angiogenic lesions induced by the inoculation of endothelial/lung carcinoma hybrid (Ea-hy 926) cells in nude mice.

To demonstrate that indinavir and saquinavir promote regression of other tumours than KS in the absence of drug pretreatment, Ea-hy 926 cells, a hybrid between H-UVEC and human lung adeno-carcinoma cells (Edgell et al., 1983), which retains most of the endothelial cell markers and is used as an angiogenic tumour model (Albini et al., 1995, Albini et al., 1996, Cai et al, 1999), have been used for in vivo studies. Mice were inoculated subcutaneously into the lower back with Ea-hy 926 cells (3×10⁶ cells/animal in 0.2 ml of 10% FBS RPMI 1640) or with the resuspension medium, all mixed with 0.2 ml of growth factor-depleted matrigel (BD Biosciences, Bedford, Mass.) prior to inoculation as described above. On the same day animal started the treatment with indinavir, saquinavir or saline solution by intragastric gavage according to the doses described in the legend to FIG. 24. Treatment was then continued for 5 days, until sacrifice. The size of the lesions present at the injection site was evaluated daily by caliper measurement. External lesion area was then calculated as the product of the two major lesion diameters. The mean size (cm²) of the lesions present at the injection site is shown.

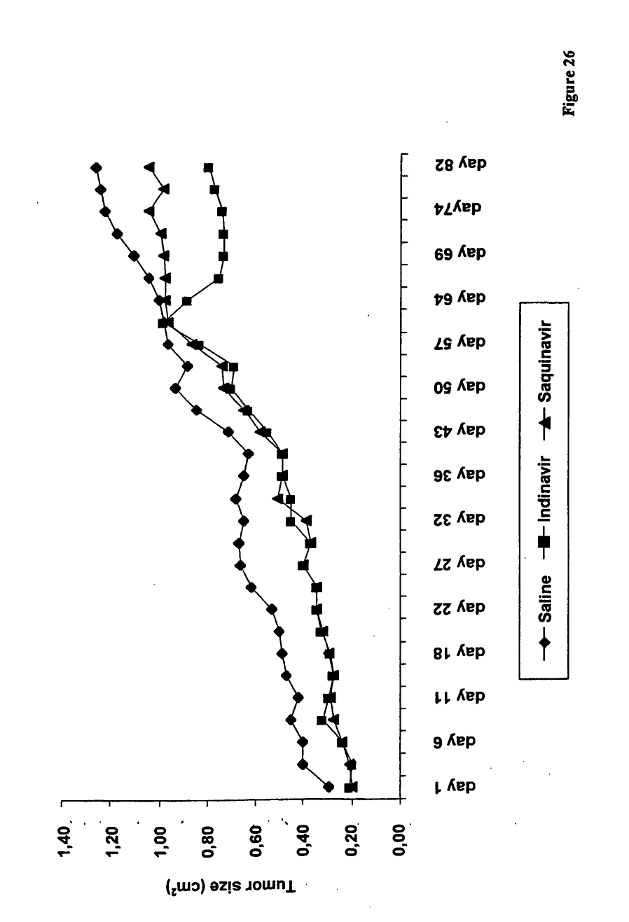

FIG. 26. Indinavir and saquinavir inhibit the development of tumour lesions induced by the inoculation of hepato-carcinoma (SK-Hep-1) cells in nude mice.

Indinavir and saquinavir are also effective in blocking the growth of tumours induced by hepato-carcinoma cells in vivo. Tumours were induced by inoculating nude mice (10 animals/group) with hepatocarcinoma cells (SK-Hep-1 cell line, obtained from ATCC, 5×10⁶ cells/site) and the animals treated daily with indinavir, saquinavir or saline solution as detailed in the key to FIG. 23. Twenty-four hours before cell injection, the mice have received sub-lethal irradiation (400 G) in order to increase the tumour up-take. The treatment with HIV-PI or saline solution continued until sacrifice. The size of the lesions present at the injection site was evaluated daily by caliper measurement. External lesion area was then calculated as the product of the two major lesion diameters. The mean size (cm²) of the lesions present at the injection site is shown.

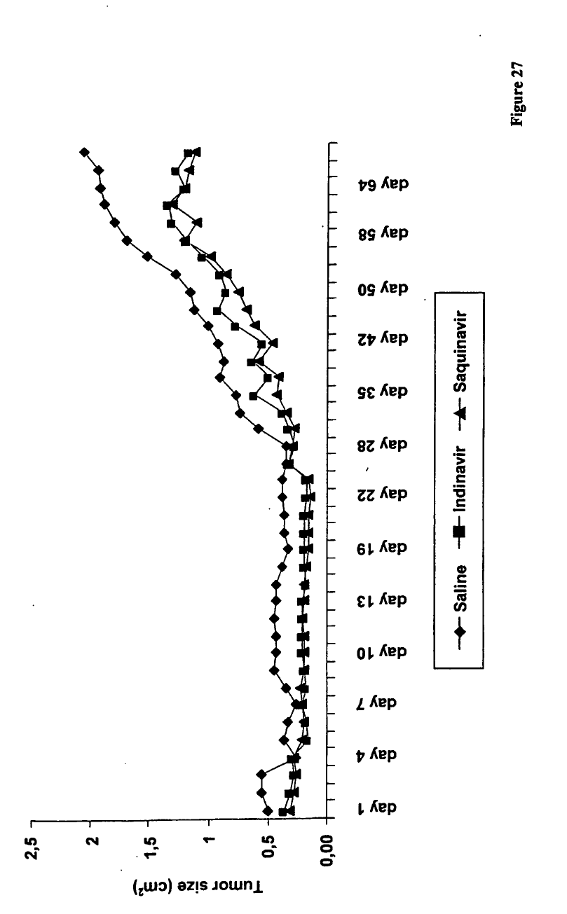

FIG. 27. Indinavir and saquinavir inhibit the development of KS-like lesions induced by the inoculation of lung carcinoma (A549) cells in nude mice.

Indinavir and saquinavir are also effective in blocking the growth of tumours induced by lung carcinoma cells in vivo. Tumours were induced by inoculating X-rated nude mice (10 animals/group) with lung carcinoma cells (A549 cell line, obtained from ATCC, 5×10⁶ cells/site) and the animals treated daily with indinavir, saquinavir or saline solution as detailed in the key to FIG. 23. The treatment with HIV-PI or saline solution continued until sacrifice. The size of the lesions present at the injection site was evaluated daily by caliper measurement. External lesion area was then calculated as the product of the two major lesion diameters. The mean size (cm²) of the lesions present at the injection site is shown.

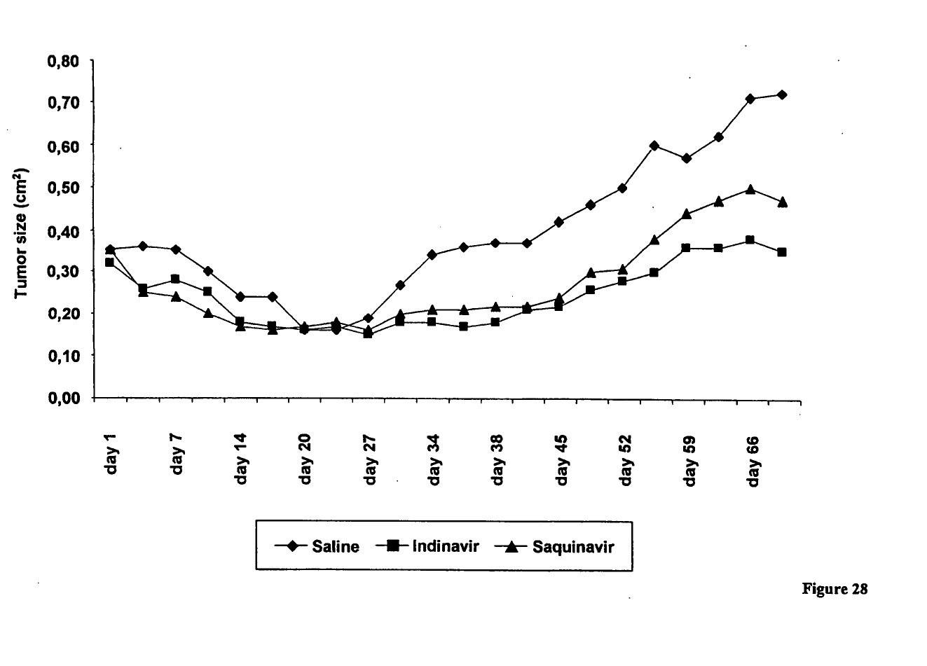

FIG. 28. Indinavir and saquinavir inhibit the development of tumour lesions induced by the inoculation of breast carcinoma (MDA-MB468) cells in nude mice. Indinavir and saquinavir are also effective in blocking the growth of tumours induced by breast adeno-carcinoma cells in vivo. Tumours were induced by inoculating X-rated nude mice (10 animals/group) with breast carcinoma cells (MDA-MBA-468 cell line, obtained from ATCC, 5×10⁶ cells/site) and the animals treated daily with indinavir, saquinavir or saline solution as detailed in the key to FIG. 23. The treatment with HIV-PI or saline solution continued until sacrifice. The size of the lesions present at the injection site was evaluated daily by caliper measurement. External lesion area was then calculated as the product of the two major lesion diameters. The mean size (cm²) of the lesions present at the injection site is shown.

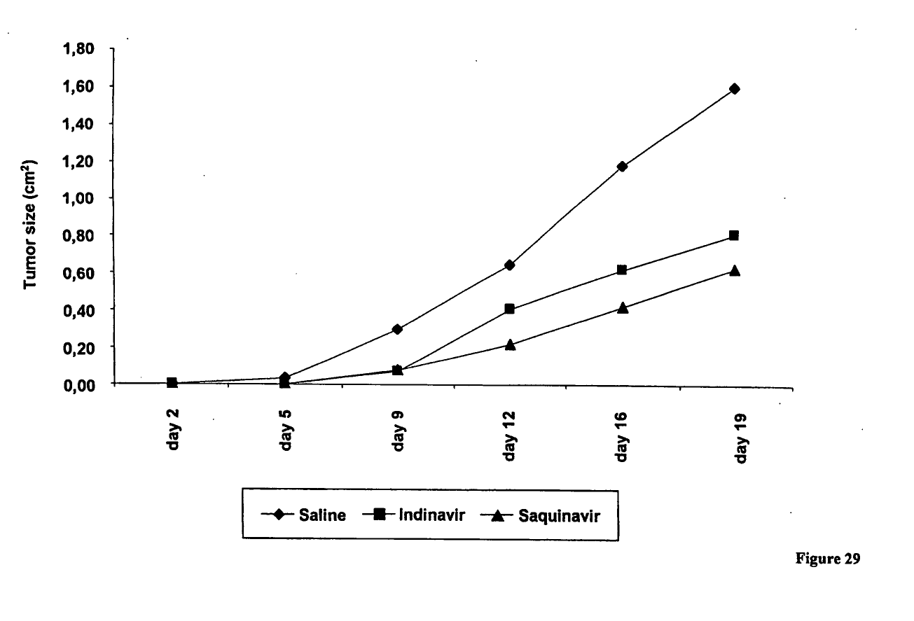

FIG. 29. Indinavir and saquinavir inhibit the development of tumour lesions induced by the inoculation of myelo-monocytic leukaemia (U937) cells in nude mice.

Indinavir and saquinavir are also effective in blocking the growth of tumours induced by myelo-monocytic leukaemia cells in vivo. Tumours were induced by inoculating X-rated nude mice (10 animals/group) with myelo-monocytic leukaemia cells (U937 cell line, obtained from ATCC, 5×10⁶ cells/site in 0.2 ml culture medium) and the animals treated daily with indinavir, saquinavir or saline solution as detailed in the legend to FIG. 23. The treatment with HIV-PI or saline solution continued until sacrifice. The size of the lesions present at the injection site was evaluated daily by caliper measurement. External lesion area was then calculated as the product of the two major lesion diameters. The mean size (cm²) of the lesions present at the injection site is shown.

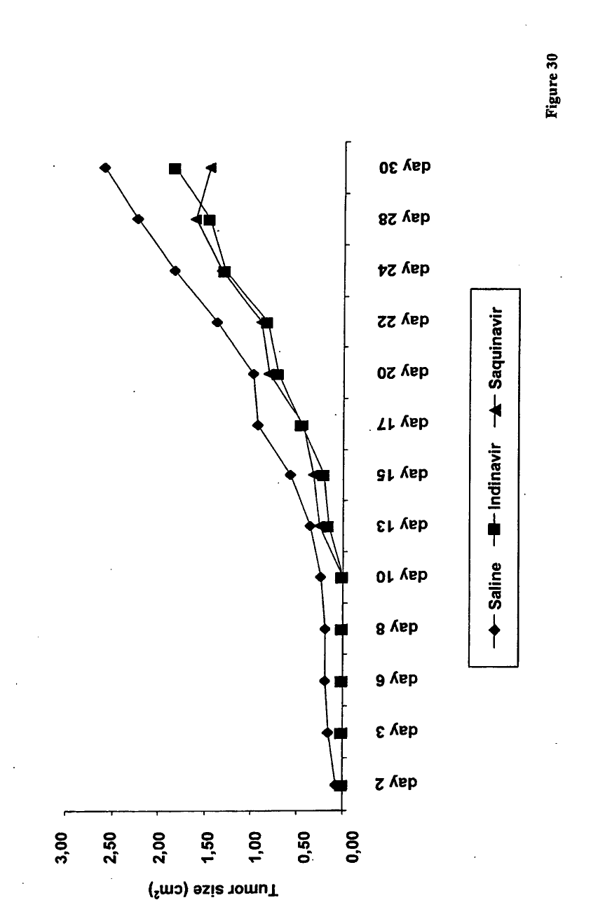

FIG. 30. Indinavir and saquinavir inhibit the development of tumour lesions induced by the inoculation of T cell leukaemia (Jurkat) cells in nude mice.

Indinavir and saquinavir are also effective in blocking the growth of tumours induced by T lymphocytic leukaemia cells in vivo. Tumours were induced by inoculating X-rated nude mice (10 animals/group) with leukaemia cells (Jurkat cell line, obtained from ATCC, 20×10⁶ cells/site) and the animals treated daily with indinavir, saquinavir or saline solution as detailed in the legend to FIG. 23. The treatment with HIV-PI or saline solution continued until sacrifice. The size of the lesions present at the injection site was evaluated daily by caliper measurement. External lesion area was then calculated as the product of the two major lesion diameters. The mean size (cm²) of the lesions present at the injection site is shown.

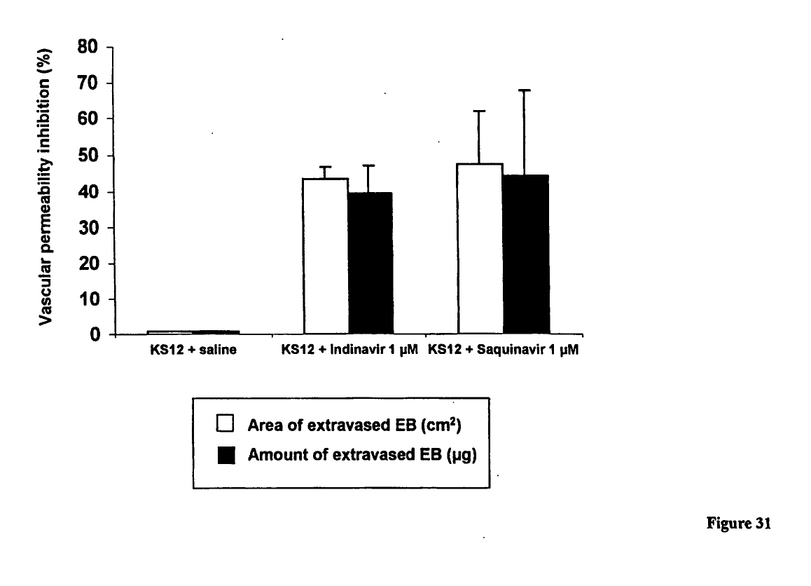

FIG. 31. Indinavir and saquinavir block the vascular permeability and oedema induced in the nude mouse by KS cells.

Nude mice were treated with Indinavir, saquinavir or saline solution (saline) for 2 days with the same doses and procedures already described. On the third day they were inoculated with pyrilamine (80 μg in 100 μl of saline solution, 4 mg/kg, Sigma), to avoid the interference of the release of histamine due to inoculation, immediately afterwards with 100 μl of Evans blue (5 mg/ml in saline solution) endovenously and then subcutaneously with KS cells (3×10⁶/mouse) cultivated in vitro in the presence of indinavir, saquinavir (1 μM) or of diluting buffer in 0.2 ml of Matrigel. As a control, each animal was inoculated controlaterally with the same volume of diluting buffer and Matrigel. After 18 hours the animals were sacrificed and the quantity of staining decanted in the inoculation site of the KS cells was measured at the level of the two largest perpendicular diameters by means of a gauge. The quantity of staining decanted was also assessed after taking skin from the inoculation site and quantified with the spectrophotometer after extraction with formamide for 24 hours at 56° C. (Nakamura et al., 1992). The quantity of staining decanted was calculated after subtraction of the optical density measured on the control site. As shown in the figure, treatment with indinavir or saquinavir reduced the quantity of staining decanted by 39.8% (p<0.05) and 44.5% (p<0.01) respectively, in the case of quantification on the spectrophotometer, and by 43.5% and 47.5% respectively in the case of measurement with a gauge.

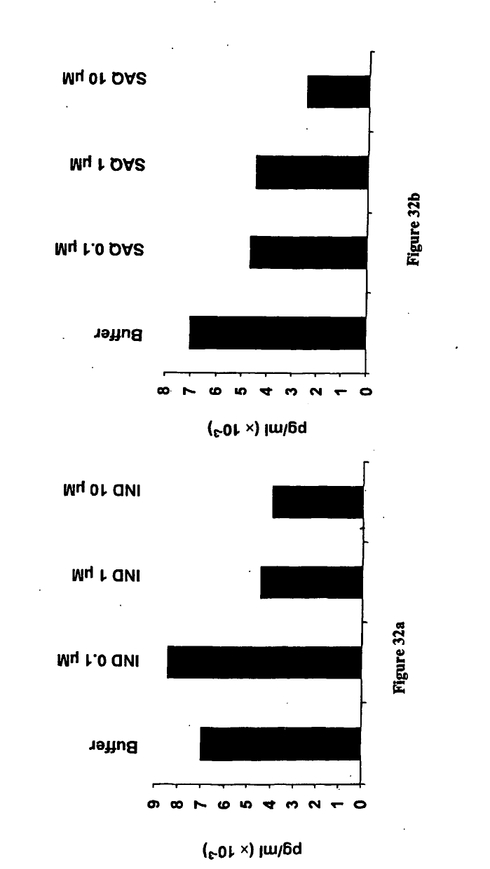

FIG. 32. Indinavir and saquinavir inhibit the production of cytokines by KS cells in vitro. Panel A: amount of IL-6 present in the supernatant of KS cells cultured in the absence (buffer) or in the presence of indinavir (IND); Panel B: amount of IL-6 present in the supernatant of KS cells cultured in the absence (buffer) or in the presence of saquinavir (SAQ).

The cells were plated in 6-well plates and cultured for 5 days as described (Ensoli et al., 1990) in the continuous presence of indinavir or saquinavir at the concentrations of 0.1, 1 and 10 μM or with dilution buffer. On the fifth day, the culture medium was replaced with a medium without serum containing bovine blood albumin (0.05% weigh/volume) in the presence of indinavir or saquinavir at the concentrations indicated. After 24 hours of incubation, the supernatants of the cultures were tested by ELISA (R & D Systems, Minneapolis, Minn., USA) to determine the quantity of IL-6 present in the medium. The quantity of IL-6 is expressed in pg/ml of supernatant. The same tests were carried out for bFGF, VEGF, IL-1α and IL-1β by means of commercial ELISA kits. Both indinavir and saquinavir reduced the production of bFGF, VEGF, IL-1α, IL-1β and IL-6.

### EXAMPLE 1

To check which of the processes required for KS development was inhibited by indinavir or by saquinavir, proliferation, migration and invasion of in response to bFGF of primary human macrovascular endothelial cells from umbelical vein cultivated in the presence or absence of scalar concentrations of indinavir or saquinavir have been performed. The concentrations of HIV-PI used were the same as those present in plasma of treated patients (Deeks et al., 1997).

As shown in FIG. 1, the HIV-PI had no effect on the basal or bFGF-induced proliferation of macrovascular endothelial cells at any of the concentrations used. Likewise, no effect was noted with indinavir or saquinavir on the survival of macrovascular endothelial cells. In contrast, both HIV-PI inhibited the migration (FIG. 2) and completely blocked the invasion of macrovascular endothelial cells (FIG. 3) promoted by bFGF at all the concentration used. The same results were obtained with primary human dermal microvascular endothelial cells (FIGS. 4 and 5) or primary human smooth muscle cells (FIGS. 6 and 7).

### EXAMPLE 2

The migration and invasion of endothelial cells are mediated by the proteolytic activity of active MMPs which degrade the basal vascular membrane allowing the endothelial cells migration and invasion, which are required for the formation of new vessels (Stetler-Stevenson, 1999). MMPs are released by endothelial cells as as zymogen proenzymes. To check whether indinavir or saquinavir have any effect on the activity of MMPs in endothelial cells, experiments were carried out to measure gelatinolytic activity with both gelatin and casein zymograms (Kleiner et al., 1993). In particular, MMP-2 is kay for both angiogenesis, tumour growth and invasion. MMP-2 zymogen (latent MMP-2, 72 kD) is proteolytically activated on the cell surface to the 64/62 kD forms by means of a complex mechanism which involves other proteases (Stetler-Stevenson, 1999). Indinavir or saquinavir (FIGS. 8 and 9 respectively) showed a minimal or no effect on the synthesis of latent MMP-2, while both HIV-PI blocked MMP-2 activation in a dose-dependent manner (FIGS. 8 and 9). These effects were observed after 24 hours of incubation of the cells with the same concentrations of HIV-PI as those present in plasma of treated patients (Deeks et al., 1997). Similar effects were also observed after 5 days of incubation with HIV-PI. To analyse the steps involved in inhibition MMP-2 activation by HIV-PI, endothelial cells were activated with a phorbol ester which is known to be very active in promoting MMP-2 conversion to its active form. As shown in FIG. 10, these experiments indicated that both HIV-PI act by inhibiting the auto-proteolytic conversion of pre-MMP2 to the active form (62 kD) (Stetler-Stevenson, 1999). Yet, the exact mechanism by which indinavir or saquinavir inhibit the conversion of the latent MMP-2 to its active form is still to be determined. In fact, no homology was found between the sequence of the active site of the protease of HIV and MMP-2 or other MMPs. However, aminoacidic sequence homology to the HIV protease catalytic site (Carr et al, 1998) was found to be present in thrombospondin, that is known to be capable of both activating or inhibiting MMPs and/or angiogenesis (Bein and Simons, 2000; Taraboletti et al., 2000). This suggests that HIV-PI may affect MMP activation or cell invasion by interacting with thrombospondin.

To study the effects of indinavir and saquinavir on other MMPs, casein zymograms were performed with endothelial cells treated with bFGF or TPA in the presence or absence of HIV-PI. These experiments showed that saquinavir was capable of inhibiting the synthesis of a casein-specific MMP induced by bFGF or TPA (FIG. 11).

Since MMPs are key for cell migration and invasion, these results indicate that indinavir and saquinavir inhibit cell migration and invasion through the inhibition of MMPs. Even though other proteases or molecules involved in cell invasion may targeted by indinavir or saquinavir, MMP-2 and casein-specific MMPs represent key examples of this effect.

### EXAMPLE 3

It has been demonstrated that MMP-2 is induced by bFGF and other angiogenic factors (Ensoli et al., 1994a; Barillari et al., 1999b; Stetler-Stevenson, 1999) and that both bFGF and MMP-2 are expressed in KS lesions (Ensoli et al., 1989; Ensoli et al., 1994a; Samaniego et al., 1998). Moreover, inhibition of bFGF or MMP activity, particularly MMP-2, is known to block angiogenesis, the formation of KS lesions and tumour growth in general (Ensoli et al., 1989; Ensoli et al., 1994a; Ensoli et al., 1994b; Stetler-Stevenson, 1999; Koivunen et al., 1999; Carmeliet and Jain, 2000). On the other hand, cell invasion is required for angiogenesis in both normal tissues and tumours. These data indicated that inhibition of cell invasion and MMPs by indinavir and saquinavir should be capable of blocking angiogenesis. Thus, the effects of indinavir and saquinavir on angiogenesis induced by bFGF and/or VEGF were studied in nude mice and in the chorioallantoic membrane (CAM) assay.

Nude mice were treated with indinavir (1.4 mg/day), saquinavir (1 mg/day) or saline solution (negative control) by means of intragastric gavage once a day for 2 days (Kleiner et al., 1993). The mice were then inoculated with bFGF (1 μg) or with its diluting buffer in the presence of matrigel (Kleiner et al., 1993; Ensoli et al., 1994a; Samaniego et al., 1998; Barillari et al., 1999a). The treatment with indinavir, saquinavir or saline solution was carried out every day for 5 more days.

The mice were then sacrificed and the inoculation areas examined both macroscopically and microscopically for the presence of KS-like angioproliferative lesions (Kleiner et al., 1993; Ensoli et al., 1994a; Samaniego et al., 1998; Barillari et al., 1999a). In agreement with the previous results (Ensoli et al., 1994a; Samaniego et al., 1998; Barillari et al., 1999a), the inoculation of 1 μg of bFGF promoted the development of angioproliferative lesions in 71% of the non-treated mice (Table 1 and FIG. 12(1)). On the contrary, treatment with indinavir or saquinavir reduced the percentage of mice that developed lesions from 28% to 25%, respectively (p<0.05) (Table 1 and FIG. 12(1)): FIG. 12(1) shows an example of these results. Treatment with indinavir or saquinavir completely blocked the formation of the lesion or greatly reduced the dimensions of the lesions. The microscopic examination of the inoculation sites in the mice treated with indinavir or saquinavir showed a marked reduction of angiogenesis and of the infiltration of the cells in comparison with mice inoculated with bFGF and not treated with HIV-PI (FIG. 12(2)). In case of total regression, the histological picture of the tissues was similar or identical to the one observed in the mice injected with the buffer alone (Negative control) (FIG. 12(2)). This was confirmed by staining with anti-FVIII or anti-CD31 antibodies and quantitation by computer-assisted analysis (Table 1). In fact, the lesional area positive for endothelial cell markers was reduced by up to 70% in HIV-PI-treated mice as compared to untreated controls (P<0.001) (Table 1).

Since VEGF co-operates synergistically with bFGF in inducing angiogenesis and KS-lesion formation, mice (6 animals/group) were also inoculated with sub-optimal amounts of bFGF (0.1 μg) and VEGF (1 μg) combined, as previously performed to observe their synergistic effect (Samaniego et al 1998), and treated with HIV-PI as indicated above. The combined addition of both factors induced lesion development in 83% of the untreated mice, however both indinavir or saquinavir reduced lesion formation to 33% and 17% (P<0.05, saquinavir) of the treated mice, respectively (Table 2). Thus, both HIV-PI inhibit the angiogenesis and the KS-like lesion development induced by the synergistic effect of bFGF and VEGF combined in nude mice. These results indicated that HIV-PI have direct anti-angiogenic effects.

To confirm that HIV-PI have direct anti-angiogenic effects, the CAM assay, which is an established in vivo assay to measure angiogenesis (Ribatti et al., 1996), was used. As shown in Table 3, indinavir or saquinavir blocked bFGF-induced angiogenesis to 42% and 19% of the untreated bFGF control, and to 36% and 11% of the untreated VEGF control, respectively (P<0.05). Notably, the block of bFGF-induced angiogenesis by both HIV-PI was comparable to that observed with taxol, a cytotoxic drug endowed with both anti-tumour and anti-angiogenic activity that is used in the therapy of KS and solid tumours (Sgadari et al., 2000) (Table 3). These data confirmed that inhibition of microvascular and macrovascular endothelial cell migration and invasion, smooth muscle cell invasion and MMP activity by HIV-PI results in the block of in vivo angiogenesis.

### EXAMPLE 4

KS cells are transdifferentiated cells of endothelial cell origin with an activated phenotype, and express both bFGF and VEGF, and MMPs (Ensoli et al., 1989; Ensoli et al., 1990; Ensoli et al., 1994a; reviewed in Ensoli and Stürzl, 1998) These data, therefore, indicate that HIV-PI may have on KS cells effects similar to those active on endothelial and smooth muscle cells, and be capable of inhibiting KS cell invasion. Thus, experiments of adhesion, proliferation, migration and invasion were carried out, cultivating the KS cells in the presence of indinavir or saquinavir at concentrations between 0.01 μM and 1 μM for 5-7 days. As shown in Table 4, indinavir or saquinavir do not inhibit the capability of the KS cells to adhere to a substrate of fibronectin. Likewise, treatment of KS cells with indinavir or saquinavir for 7 days had no effect on cell proliferation measured by counting viable cells by trypan blue dye exclusion (Table 4).

To determine whether HIV-PI interfere with the capacity of KS cells to migrate and invade the basal membrane in response to angiogenic factors, KS cells treated for 5 days with indinavir or saquinavir (0.01 μM-1 μM) were placed in the upper compartment of Boyden chambers always in the presence of HIV-PI, while bFGF was placed in the lower compartment as a chemo-attractant. As shown in Table 4, neither indinavir nor saquinavir had any effect on the migration of KS cells. In contrast, both drugs inhibited KS cell capability of invading a matrigel substrate in a dose-dependent manner. In particular, both HIV-PI inhibit the invasion of KS cells by 30-40% (p<0.05) (Table 4 and FIG. 13).

These data indicated that the mechanism underlying the effects of HIV-PI on cell invasion in response to chemotactic stimuli, including—for example—inhibition of MMPs, is most likely active on many cell types including tumour cells.

### EXAMPLE 5

To verify whether HIV-PI are capable of specifically inhibiting invasion of tumour cells, experiments of cell proliferation and invasion were carried out on tumour cell lines obtained from tumours of various origin. In particular, the following cell lines were studied: Ea-hy 926, derived from a hybrid between H-UVEC and human lung carcinoma cells (Edgell et al, PNAS 1983), hepato-carcinoma cells (SK-Hep-1), lung carcinoma cells (A549), breast adeno-arcinoma cells (MDA-MB468), and myelo-monocytic leukaemia cells (U937). Indinavir or saquinavir showed no significant effects on proliferation of these cell lines (FIGS. 14, 16, 18, 20). In contrast, both HIV-PI significantly inhibited tumour cell invasion at the same concentrations as those present in sera from treated patients (FIGS. 15, 17, 19, 21, 22). Therefore, these data indicated that the capability of HIV-PI of inhibiting invasion of normal and neoplastic cells underlies a mechanism that is common to all cells, such as the block of molecules and enzymes involved in cell migration and invasion including, in particular, MMPs.

### EXAMPLE 6

These results indicated that, despite lack of effects on tumour cell proliferation, HIV-PI may be capable of inhibiting tumour growth by selectively blocking tumour cell invasion and tumour angiogenesis, which are both required for the development of tumours, tumour infiltration and metastasis (Carmeliet et al., 2000). Therefore, in vivo studies were performed to determine whether HIV-PI were effective in inhibiting the growth of xenograft tumour models including the tumour cell lines used in in vitro studies.

Firstly, the effects of indinavir or saquinavir have been studied on the formation of KS-like lesions promoted by the inoculation of primary human KS cells in nude mice, a model in vivo widely used in preclinical studies of the efficiency of anti-KS therapies (Ensoli et al., 1994b; Koivunen et al., 1999; Sgadari et al., 2000). These angioproliferative lesions are transient, of murine origin and are developed in response to cytokines, such as bFGF and VEGF, IL-1, IL-6 and others, released by the KS cells (Ensoli et al., 1989; Ensoli et al., 1994a; Ensoli et al., 1994b; Fiorelli et al., 1995; Samaniego et al., 1995; Samaniego et al., 1997; Sgadari et al., 2000). In fact, at least in initial phase, KS is a reactive angioproliferative diseases and not a true neoplasm (Ensoli and Sturzl, 1998). The animals were treated with the same procedures and doses of indinavir or saquinavir used in the experiments illustrated above. As shown in Table 5, the inoculation of KS cells induced the formation of KS-like lesions in 100% of the animals. Treatment with indinavir or saquinavir reduced the percentage of mice that developed a lesion to 43% and 25% respectively. Macroscopically, lesions were florid and highly vascularised in untreated mice, but smaller, pail and in a regressing phase in HIV-PI-treated animals. Similarly, in untreated mice lesions had intense neovascularization, spindle cell infiltration, oedema and red blood cell extravasation (FIG. 23). In contrast, HIV-PI-treated mice showed a large necrotic area at the site of cell injection, involving up to 85% of the whole lesional area, and a marked reduction of both neo-formed vessels and spindle cell infiltration, which were mostly confined at the periphery of the necrotic/regressing area (FIG. 23).

To determine whether HIV-PI can also promote KS regression in the absence of any drug pre-treatment, experiments were also performed by treating mice with HIV-PI at the time of KS cell inoculation. As shown in FIG. 24, KS lesions generally show a slow regression upon time, however, in HIV-PI-treated mice lesions regressed much faster and, at sacrifice, the external lesional area was similar or identical to that of the negative controls (P<0.001).

The Tat protein of HIV increases the frequency and aggressiveness of KS in subjects infected with HIV-1 (Ensoli et al., 1994a). This is due to the induction by Tat of the adhesion, migration, invasion and proliferation of endothelial cells and of KS. In fact Tat synergistically increases the effects of bFGF on angiogenesis and on KS (Ensoli et al., 1994a; Barillari et al., 1999a and 1999b). However, Tat requires the presence of bFGF or of inflammatory cytokines to exert its action on KS, since they increases the expression of the receptors for Tat on the cells and on the tissues (Barillari et al., 1992; Barillari et al., 1993; Albini et al., 1995; Fiorelli et al., 1995; Fiorelli et al., 1999; Barillari et al., 1999a; Barillari et al., 1999b).

Therefore, to check whether HIV-PI inhibit the effect of Tat and bFGF combining on angiogenesis and on KS, nude mice were inoculated with bFGF and Tat and treated with indinavir, saquinavir or with the buffer used for re-suspending them. As shown in Table 6, both indinavir and saquinavir reduced the percentage of nude mice that developed KS lesions (50% and 20% respectively).

These results indicated that HIV-PI are capable of inhibiting the development and induce the regression of a reactive, hyperplastic tumour model like KS (Ensoli and Sturzl, 1998) despite lack of effects on KS cell proliferation, due to their effects on cell invasion, MMP activity, and angiogenesis.

### EXAMPLE 7

To determine whether HIV-PI could inhibit the growth of a malignant angiogenic tumour, nude mice were inoculated with the EA-hy 926 cell line. This cell line is derived from a hybrid between H-UVEC and human lung adeno-carcinoma cells (Edgell et al, PNAS 1983), retains most of the endothelial cell markers and is used as an angiogenic tumour model (Albini et al, Nat Med 1996; Albini et al, PNAS 1995; Cai et al, Lab Invest 1999). HIV-PI were administered to nude mice starting 2 days prior to tumour cell inoculation. As shown in Table 7, tumours arose in 83% of untreated mice but only in 33% and 25%, respectively, of mice treated with indinavir or saquinavir (P<0.05). Similarly, the external tumour area was reduced in mice treated with both HIV-PI (P<0.05), reaching the size of the negative controls (Table 7). Residual tumours in treated animals showed a strong reduction of both tumour growth and angiogenesis as determined histologically and by staining with anti-FVIII-RA or anti-CD31 antibodies as compared to controls (P<0.001) (Table 7). Inhibition of tumour growth was also observed in the absence of drug pre-treatment (FIG. 25). In these animals, in fact, the external tumour size at sacrifice was reduced by more than 50% as compared to untreated controls (P<0.001) (FIG. 25). Thus, HIV-PI inhibit the in vivo growth of an angiogenic tumour model by blocking directly tumour cell invasion and angiogenesis despite the lack of effects on EA-hy 926 cell proliferation.

### EXAMPLE 8

To determine whether HIV-PI could inhibit the growth of malignant solid and lymphoid tumours, nude mice were inoculated with hepato-carcinoma cells (SK-Hep-1), lung carcinoma cells (A549), breast adeno-carcinoma cells (MDA-MB-468), myelo-monocytic leukaemia cells (U937), and T lymhocytic leukaemia cells (Jurkat). The growth of all these xenograft tumours was significantly inhibited by both indinavir or saquinavir despite the lack of effect of HIV-PI on the proliferation of these cell lines (FIGS. 26-30). Thus, these data indicate that block of tumour and endothelial cell invasion due to inhibition of MMP activity by HIV-PI is responsible for the effects of these drugs on tumour growth.

### EXAMPLE 9

Since MMPs are involved in vascular permeability and oedema formation (Carmeliet et al, 2000), which are important clinical features angiogenesis, KS, tumours and inflammatory diseases, experiments of vascular permeability were carried out in nude mice inoculated with KS cells. These cells induce oedema because they produce cytokines with oedemigenic effects including VEGF, bFGF (in combination with VEGF), IL-1, IL-6, and others. Nude mice were treated with indinavir, saquinavir or saline solution for 2 days according to the doses and procedures already described in example 6, inoculated endovenously with Evan's blue and then injected with KS cells cultivated in vitro in the presence of indinavir or saquinavir (1 μM) or of dilution buffer. After 12 hours the animals were sacrificed, the stained area present on the site of inoculation of the KS cells was measured with a caliper and the extravasated dye was extracted from tissues with formamide and measured by spectrophotometry (Nakamura, Science 1992). As shown in FIG. 31, treatment with indinavir or saquinavir reduced the amount of extravasated dye by 39.8% (p<0.05) and 44.5% (p<0.01) respectively, and the stained area by 43.5% and 47.5%, respectively (FIG. 31).

### EXAMPLE 10

KS cells secrete cytokines with an inflammatory, oedemigenic, angiogenic and proliferative activity with autocrine and paracrine effects (Ensoli and Stürzl, 1998). These factors mediate all the processes required for KS-like lesions formation (angiogenesis, cellular proliferation and invasion, inflammatory Infiltration, oedema) and vascular permeability and oedema induced by KS cells in nude mice. To determine the effects of indinavir and saquinavir on the production of cytokines, KS cells were cultivated in the presence or absence of scalar concentrations of indinavir or saquinavir. The quantity of bFGF, VEGF, IL-1 and IL-6 was dosed with immuno-enzymatic trials in the supernatants of the KS cells after 24 hours of culture in the absence of serum and in the continuous presence of the two HIV-PI (ELISA). Indinavir and saquinavir inhibited the production of IL-1α, IL-1β and IL-6 by KS cells. As an example of these effects, FIG. 32 shows the inhibition of IL-6, a typical inflammatory cytokine produced by KS cells and endothelial cells, but also by lymphocytes and monocytes of the blood and of the tissues and which also has angiogenic effects (Mateo et al., 1994; Cohen et al., 1996). Furthermore IL-6 plays a key role in the multicentric Cstleman's diseases and in the growth of lymphomas (Tosato et al., 1993; Peterson and Frizzera, 1993; Asou et al., 1998; Ramsay et al., 1994), another type of tumour whose incidence is reduced in patients treated with HIV-PI (International Collaboration on HIV and Cancer 2000).

Discussion

These results indicate that HIV-PI have specific inhibitory effects on cell migration and/or invasion but not cell proliferation. These effects appear to be due to a mechanism that is general for many primary and tumour cell types of different origin, and target key molecules intervening in cell migration and invasion that are not related to the cell proteasome. For example, our studies show the effects of HIV-PI are related to inhibition of MMP activation or production and may target other molecules involved in MMP metabolism such as, for example, thrombospondin. Due to these activities, HIV-PI are capable of blocking several cell processes requiring cell migration, invasion, and/or MMP activity, including angiogenesis, vascular permeability, oedema formation and growth of both reactive hyperplastic tumours, such as KS, or malignant solid or lymphoid neoplasms. Since migration of inflammatory and immune cells also require cell invasion and MMP activity, our data indicate that HIV-PI can inhibit tissue cell infiltration during inflammatory or immune responses. Moreover, HIV-PI inhibit the production of cytokines and other factors which mediate the formation of KS and the growth of other tumours and the inflammatory infiltration associated with them.

HIV-PI also have anti-inflammatory effects as they reduce the production of cytokines such as IL-6, IL-1, and probably other cytokines involved in the inflammation and which are also present in human or mouse KS lesions. These same inflammatory cytokines are able to induce the production of angiogenic factors (bFGF, VEGF) and also have angiogenic effects in vivo (Barillari et al., 1992; Samaniego et al., 1995; Fiorelli et al., 1995; Samaniego et al., 1997; Samaniego et al., 1998; Fiorelli et al., 1998; Fiorelli et al., 1999; Barillari et al., 1999a). In particular, IL-6 plays a key role in the multicentric Castleman's diseases and in the growth of lymphomas (Tosato et al., 1993; Peterson and Frizzera, 1993; Ramsay et al., 1994; Asou et al., 1998).

HIV-PI bind to the active site of HIV protease, which belongs to the family of aspartyl-proteases. It has recently been demonstrated that these drugs can inhibit an fungal aspartyl-protease (Cassone et al., 1999). However, none of the known proteases which are involved in cell migration and invasion is an aspartyl protease, and no sequence homology was found between the active site of the HIV protease and the proteases involved in these processes except thrombospondin. The effects that we demonstrated on cell migration, invasion, and MMPs were therefore completely unforeseeable and could not have been expected.

In fact, although some studies suggested that HIV-PI have an effect on the cell metabolism, proteasome and immunity (Deeks et al., 1997; André et al., 1998; Weichold et al., 1999; Ledru et al., 2000; Tovo, 2000, patent appl.n WO99/63998, patent appl.n WO0033654), we have demonstrated that HIV-PI exert a direct anti-angiogenic, anti-tumour, anti-oedemigenic and anti-inflammatory activity which is not connected with known aspartyl-proteases, cell proteasome, or effects of HIV-PI on the replication of HIV or of HHV-8. In fact, the models in vitro and in vivo of cell proliferation, migration, invasion, angiogenesis, KS and tumours used in this study are free of infective agents.

The same results were obtained with both indinavir and saquinavir, which share a similar structure with the other HIV-PI, though with specific chemical substituents for each drug. So these data indicate that the activities of HIV-PI that we discovered for indinavir and saquinavir are a property common to all HIV-PI, which is in agreement with the common effects of different HIV-PI observed in treated individuals (Lebbé et al., 1998; Cattelan et al., 1999; International Collaboration on HIV and Cancer, 2000).

The above effects of HIV-PI are observed at the same drug concentrations present in the plasma of treated patients, which are well tolerated by these individuals. Likewise, no toxic effects of indinavir or saquinavir were observed in vitro or in mice. Although previous data showed that HIV-PI inhibit the cell proteasome and that proteasome inhibition induces programmed cell death in proliferating endothelial cell (Andre et al., 1998, Hannes et al, 2000), in our studies indinavir and saquinavir did not show any effects on cell survival or growth in endothelial cells, smooth muscle cells or tumour cells. In contrast, they selectively inhibited cell migration and invasion, suggesting that the HIV-PI do not damage pre-existing vessels or tissues. Since cell motility and angiogenesis are essential not only for the development of KS, but also for the growth and metastasis of tumours (Carmeliet and Jain, 2000; Stetler-Stevenson, 1999), the results described so far indicate that HIV-PI are promising anti-angiogenic and anti-tumour drugs. Moreover, the same results indicate that HIV-PI block vascular permeability and inflammation induced by inflammatory cytokines and vascular permeability factors, and the production of cytokines with a key role in multicentric Castleman's disease, and in the growth of lymphomas (Tosato et al., 1993; Peterson and Frizzera, 1993; Ramsay et al., 1994; Asou et al., 1998). HIV-PI and drugs similar to or derivatives from them could therefore be exploited to block the angiogenesis, growth, invasion and metastasis of solid tumours and tumours of the blood, oedema and inflammation, and could thus be successfully used in the therapy of KS, of tumours, of angioproliferative diseases, and inflammatory and autoimmune diseases both in HIV-negative subjects and in subjects infected by HIV.

TABLE 1
Indinavir and saquinavir block bFGF-induced angiogenic
KS-like lesions in nude mice.

| | | No. of mice with | | |
|---|---|---|---|---|
| | | macroscopic | | |
| | | vascular | Tissue staining | |
| | | lesions/No. of | (area % ± SD) | |
| Treatment | Injection | inoculated mice (%) | FVIII-RA | CD31 |
| Saline | Buffer | 0/22 (0) | 0.20 ± 0.2 | 0.06 ± 0.1 |
| Saline | bFGF | 20/28 (71) | 4.32 ± 2.0 | 3.45 ± 1.7 |
| Indinavir | bFGF | 8/28 (28) | 1.32 ± 1.2 | 1.00 ± 0.6 |
| Saquinavir | bFGF | 7/28 (25) | 1.13 ± 0.6 | 0.83 ± 0.3 |

The nude mice were inoculated with bFGF (1 μg) to induce the formation of KS-like angioproliferative lesions or with its re-suspension buffer (control) and treated with indinavir, saquinavir or saline solution. At the time of sacrifice, the inoculation sites were examined to check for the presence of macroscopic angioproliferative lesions and analyzed microscopically after haematoxilin & eosin (H&E) staining or frozen for histochemical analysis of endothelial the cell markers FVIII-RA and CD31. Here are listed the number (No.) of mice that developed lesions with respect to the number (No.) of mice inoculated, and the percentage (%) of mice that developed lesions. The reduction of the number of KS-like lesions in the treated animals is statistically significant (standard test for the proportions, p<0.05). Angiogenesis was quantitated by determining the tissue area stained for FVIII-RA or CD31. Shown are the percentages [mean±standard deviations (SD)] of lesional area stained for FVIII-RA or CD31 that were quantitated as described below. The decrease of FVIII or CD31 expression in residual lesions from HIV-PI-treated animals was statistically significant (P<0.001).

To perform these in vivo experiments the same formulas of indinavir (Merck Sharpe & Dhome Ltd., Haarlem, N L) or saquinavir (Roche, Hertfordshire, G B) used in patients infected with HIV were dissolved in a saline solution and administered to nude mice (Balb/c nu/nu females, Charles River, Calco, Italy, 5-6 weeks old) by intragastric gavage. To test their toxicity, indinavir and saquinavir were administered once a day for a total of 8 days in doses of 35, 70 or 17.5 mg/Kg/day or of 18, 36 or 9 mg/Kg/day respectively, in a volume of 0.4 ml. These doses correspond, respectively, to the whole dose, double or half the dose of HIV-PI used daily in patients infected with HIV (Deeks et al., 1997). No organ toxicity or systemic toxicity was observed for any of these doses. The mice were treated with 70 mg/Kg/day of indinavir (corresponding to 1.4 mg/day) or with 36 mg/Kg/day of saquinavir (corresponding to 1 mg/day) once a day for a total of 7 days, starting from two days prior to the inoculation of bFGF. The control animals were treated with the same volume of saline solution. On the third day the mice were inoculated subcutaneously at the level of the lower dorsal quadrant with 1 μg of recombinant bFGF (Roche, Mannheim, Germany) diluted in 0.2 ml of phosphate buffer (PBS)-0.1% bovine blood albumin (BSA) or with its re-suspending buffer, mixed with 0.2 ml of Matrigel (Collaborative Biomedical Products, Bedford, Mass.) prior to inoculation, as described previously (Ensoli et al., 1994a). Four days later the mice were sacrificed, the inoculation sites were examined to check for the presence of macroscopic KS-like angioproliferative lesions, and sections of tissue histologically examined after staining with H&E. For immunohistochemical analysis, frozen tissue sections were fixed with cold acetone and stained with rabbit anti-human FVIII-RA polyclonal antibodies (Ab) (Dako, Glostrup, Denmark; 1:2000 dilution) or anti-mouse CD31 rat monoclonal Ab (BD Biosciences; 1:1000 dilution). Digital images (200× magnification) were captured by a color CCD camera (Zeiss) and analyzed by acquiring 4-9 microscopic fields (about 0.15 mm² per field) corresponding to the whole histologic sections. Staining was quantitated by the KS300 (Zeiss) image analysis software and expressed as the percentage of positive area over the total tissue area.

TABLE 2
Indinavir or saquinavir inhibit the development of angiogenic-KS
like lesions induced by bFGF and VEGF combined.

| | | No. of mice developing |
|---|---|---|
| Angiogenic factors | Treatment | lesions/No. of mice |
| bFGF 0.1 μg | Saline | 2/4 (50%) |
| VEGF 1 μg | Saline | 0/6 (0%) |
| bFGF 0.1 μg + VEGF 1 μg | Saline | 5/6 (83%) |
| | Indinavir | 2/6 (33%) |
| | Saquinavir | 1/6 (17%) |

Nude mice were inoculated with buffer, bFGF, VEGF (R&D systems, Minneapolis, Minn.), or bFGF and VEGF combined in matrigel and treated with indinavir, saquinavir or saline solution as detailed in key to Table 1. At sacrifice the sites of injection were examined for the presence of macroscopic angioproliferative lesions and tissue stained by H & E and analysed microscopically. Here are listed the number (No.) of mice that developed lesions with respect to the number (No.) of mice inoculated, and the percentage (%) of mice that developed lesions. The number of mice developing angiogenic KS-like lesion was reduced upon treatment with both indinavir or saquinavir (standard test for proportions, P<0.05).

TABLE 3
Effects of indinavir or saquinavir on angiogenesis induced by
bFGF or VEGF in the chorioallantoic membrane assay (CAM).

| | | Average vessel |
|---|---|---|
| Angiogenic factor | Treatment (n. of eggs) | (number/mm² ± SD) |
| Buffer | Saline (23) | 4.39 ± 1.12 |
| bFGF | Saline (11) | 13.50 ± 3.03 |
| | Indinavir (11) | 8.22 ± 2.54 |
| | Saquinavir (13) | 6.08 ± 1.84 |
| VEGF | Saline (8) | 13.51 ± 2.42 |
| | Indinavir (9) | 7.70 ± 2.52 |
| | Saquinavir (7) | 5.39 ± 0.67 |
| bFGF | Taxol (4) | 7.71 ± 1.63 |

CAM assays were performed with 1 mm³ sterilized gelatin sponges (Gelfoam; Upjohn Co, Kalamazoo, Mich.) adsorbed with bFGF or VEGF (1 μg or 100 ng, respectively) in 5 μl of PBS, and with buffer, HIV-PI (10 μM) or taxol (250 nM) (Bristol-Myers Squibb Co., Princeton, N.J.) as described (Ribatti et al, Int. J. Dev. Biol., 1996). CAM were examined daily until day 12 under an Olympus SZX9 stereomicroscope. Images (1024×1024 pixels) were captured at a distance of 2 mm from the edge of the sponge using a cooled digital CCD Hamamatsu ORCA camera (Hamamatsu Photonics Italia, Arese, Italy). Vessel number was quantified with the ImageProPlus 4.0 imaging software (Media Cybernetics, Silver Spring, Md.) in 3 randomly selected areas per egg (1 mm³).

Results are expressed as the average number of vessels/mm²±SD from the indicated number of CAM per experimental condition. The doses of indinavir, saquinavir or taxol utilised are similar to the drug concentrations present in plasma of treated patients (Sonnichsen, D. S. & Relling, M. V., Clin. Pharmacokinet. 1994, Deeks, S. G et al, JAMA 1997). Administration of indinavir, saquinavir or taxol alone was not associated with toxicity and did not affect basal CAM vascularization (data not shown). The inhibition of vessels formation by indinavir or saquinavir was statistically significant (ANOVA and Student-Newman-Keuls test; P<0.05).

TABLE 4
Effects of indinavir and saquinavir on KS cells in vitro.

| | Indinavir | | | Saquinavir | | |
|---|---|---|---|---|---|---|
| | (μM)* | | | (μM)* | | |
| KS Cells | 0.01 | 0.1 | 1 | 0.01 | 0.1 | 1 |
| Adhesion | ND | ND | 1.05 | ND | ND | 1 |
| Proliferation | 1 | 1.08 | 1.11 | 1 | 1.15 | 1.25 |
| Migration | ND | ND | 1.03 | ND | ND | 1.21 |
| Invasion | 0.98 | 0.74** | 0.71** | 1 | 0.79** | 0.62** |

*Fold expression increase as compared to control (1 fold)
**p < 0.05
ND, not determined

Adhesion, proliferation, migration and invasion assays were carried out cultivating KS cells in the presence of indinavir or saquinavir at concentrations between 0.01 and 1 μM for 5-7 days. Indinavir or saquinavir do not inhibit KS cell capability of adhering to a substrate of fibronectin. Likewise, treatment of the KS cells with indinavir or saquinavir for 7 days has no effect on cell proliferation as measured by trypan blue dye exclusion.

Neither indinavir nor saquinavir had any effect on the migration of KS cells. In contrast, both the drugs Inhibited Ks cell capability of invading a matrigel membrane in a dose-dependent manner (p<0.05).

Migration and invasion assays were carried out essentially as detailed in the key to FIGS. 2 and 3. KS cells cultured for 5 days in the presence of indinavir or saquinavir (0.01 μM-1 μM) or dilution buffer were placed in the upper compartment of Boyden chambers always in the presence of HIV-PI, while bFGF was placed in the bottom compartment as a chemo-attractant.

TABLE 5
Effects of indinavir or saquinavir on angioproliferative KS-like
lesions induced by the inoculation of KS cells in nude mice.

| | | No. of mice with |
|---|---|---|
| | | macroscopic lesions/No. of |
| Treatment | Injection | inoculated mice (%) |
| Saline | Medium | 0/8 (0) |
| Saline | KS cells | 14/14 (100) |
| Indinavir | KS cells | 7/16 (43) |
| Saquinavir | KS cells | 4/16 (25) |

The nude mice were inoculated with KS cells (3×10⁶) to induce the formation of KS-like angioproliferative lesions or with its re-suspension buffer (control) and treated with indinavir, saquinavir or saline solution according to the doses and procedures described in the key to Table 1, starting 2 days before cell inoculation until sacrifice, 5 days later. At the time of sacrifice, the sites of inoculation were examined to check for the presence of macroscopic angioproliferative KS-like lesions as described in the key to Table 1. Here are listed the number (No.) of mice that developed lesions with respect to the number (No.) of mice inoculated, and the percentage (%) of mice that developed lesions. The decrease of angiogenic KS-like lesion formation in HIV-PI-treated animals was statistically significant (standard test for proportions, P<0.001). The histological picture of the inoculation sites is shown in FIG. 23.

TABLE 6
Indinavir and saquinavir block the formation of angioproliferative
KS-like lesions promoted by the inoculation of bFGF and HIV-1
Tat combined in nude mice

| | | No. of macroscopic vascular |
|---|---|---|
| Injection | Treatment | lesions/No. of mice injected (%) |
| Buffer | Saline solution | 0/18 (0%) |
| BFGF + Tat | Saline solution | 7/10 (70%) |
| BFGF + Tat | Indinavir | 5/10 (50%) |
| BFGF + Tat | Saquinavir | 2/10 (20%) |

Nude mice were inoculated with bFGF (1 μg) and Tat (10 μg) combined to induce the formation of KS-like angioproliferative lesions or with its re-suspending buffer (control) and treated with indinavir, saquinavir or saline solution. At the time of sacrifice, the inoculation sites were examined to check for the presence of macroscopic angioproliferative lesions. Here are listed the number (N.) of mice that developed lesions with respect to the number (N.) of mice inoculated, and the percentage (%) of mice that developed lesions.

The mice have been treated with indinavir and saquinavir according to the procedures and doses described in Table 1 for a total of 7 days, starting from two days prior to the inoculation of bFGF and Tat. The control animals were treated with the same volume of saline solution. On the third day the mice were inoculated subcutaneously at the level of the lower dorsal quadrant with 1 μg of recombinant bFGF and 10 μg of HIV-1 Tat diluted in 0.2 ml of PBS-0.1% BSA or with its re-suspending buffer, mixed with 0.2 ml of Matrigel prior to inoculation, as described previously (Ensoli et al, Nature 1994). Four days later the mice were sacrificed, the inoculation zones were examined to check for the presence of macroscopic KS-like angioproliferative lesions, and tissue sections examined microscopically after staining with haematoxylin/eosin.

TABLE 7
Indinavir or saquinavir inhibit the development of
angiogenic tumours induced by the inoculation
EA-hy 926 cells into nude mice.

| | | No. of | | | |
|---|---|---|---|---|---|
| | | mice with | | | |
| | | macroscopic | | | |
| | | lesions/No. | External | Tissue | |
| | | of | lesion | staining | |
| | Injec- | inoculated | size | (area % ± SD) | |
| Treatment | tion | mice (%) | (cm² ± SD) | FVIII-RA | CD31 |
| Saline | Medium | 0/8 (0) | 0.58 ± 0.15 | 0 | 0 |
| Saline | cells | 10/12 (83.3) | 0.71 ± 0.2 | 2.91 ± 1 | 2.30 ± 1.7 |
| Indinavir | cells | 4/12 (33.3) | 0.63 ± 0.17 | 1.01 ± 1.5 | 0.70 ± 0.6 |
| Saquinavir | cells | 3/12 (25) | 0.55 ± 0.14 | 0.71 ± 0.8 | 0.08 ± 0.1 |

Nude mice were injected subcutaneously with EA-hy 926 cells (3×10⁶ cells/site) (see key to FIG. 25) and treated by intragastric gavage with indinavir, saquinavir or saline solution since 2 days prior to cell inoculation as described above (see key to Table 1). Mice were sacrificed 5-6 days after cell injection and lesions present at the injection site measured by caliper, and analyzed microscopically after H&E staining or by immuno-histochemical analysis. The decrease of angiogenic tumour lesion formation in HIV-PI-treated animals was statistically significant (standard test for proportions, P<0.05). Tumour-associated angiogenesis was evaluated after immuno-histochemical staining for FVIII-RA or CD31 markers, as described in legend to Table 1. Shown are external lesion size (cm²±SD) and the percentage of stained tumour tissue (±SD) as quantified by the KS300 image analysis software (Zeiss) (see methods). The residual lesional area still present at sites of injection were smaller in size in both mice treated with indinavir or saquinavir (P<0.05) and showed a lower expression of both FVIII or CD31 (P<0.001).

### REFERENCES

Albini A. et al. Proc. Natl. Acad. Sci. USA 92, 4838 (1995).
Albini A. et al., Nat. Med. 2, 1371 (1996)
André P. et al., Proc. Nat. Acad. Sci. USA 95, 13120 (1998).
Asou H. et al., Blood 91, 2475 (1998).
Barillari G. et al., J. Immunol., 149, 3727 (1992).
Barillari G. et al., Proc. Natl. Acad. Sci. USA 90, 7941 (1993).
Barillari G. et al., J. Immunol., 162, 1165 (1999a).
Barillari G. et al., Blood 94, 663 (1999b).
Bein K and Simons M., J. Biol. Chem. 275, 32167 (2000)
Blum L. et al., AIDS 11, 1653 (1997).
Cai T. et al., Lab Invest 79, 1151 (1999) Carmeliet P., R. K. Jain, Nature 14, 249 (2000).
Carr A et al., Lancet 351, 1881 (1998).
Cassone A. et al., J. Infect. Dis. 180, 448 (1999).
Cattelan A. M. et al., Eur. J. Cancer 35, 1809 (1999).
Cohen T. et al., J. Biol. Chem. 271, 736 (1996).
De Milito A. et al., J. Med. Virol. 57, 140 (1999).
Deeks S. G. et al., JAMA 2, 145 (1997).
Edgell, C. J., et al., PNAS 80, 37341983
Ensoli B. et al., Science 243, 223 (1989).
Ensoli B. et al., Nature 345, 84 (1990).
Ensoli B. et al., Nature 371, 674 (1994a).
Ensoli B. et al., J. Clin. Invest. 94, 1736 (1994b).
Ensoli B., M. Stürzl, Cytokine Growth Factor Rev. 9, 63 (1998).
Fiorelli V. et al., J. Clin. Invest. 95, 1723 (1995).
Fiorelli V. et al., Blood 91, 956 (1998).
Fiorelli V. et al., J. Immunol. 162, 1165 (1999).
International Collaboration on HIV and cancer, J. Natl. Cancer Inst. 92, 1823 (2000).
Fujimoto N, et al., Clin Chim Acta 221, 91 (1993)
Gruber A et al., J. Biol. Chem. 276, 47840 (2001).
Hannes C A et al, FASEB J. 14, 85 (2000)
Kleiner D. E., W. G. Stetler-Stevenson, Anal, Biochem. 218, 325 (1993).
Koivunen E. et al., Nat. Biotechnol. 17, 768 1999).
Lebbé C. et al., AIDS 12, 45 (1998).
Ledru E. et al., Blood 95, 3191 (2000).
Leppert D et al., Brain 121, 2327 (1998).
Leppert D et al., Clin Infect Dis 31, 80 (2000).
Masood R. Z. Y. et al., Blood 85, 3423 (1995).
Mateo R. B. et al., Am. J. Physiol. 266, R1840 (1994).
Meade-Tollin L. C. et al., Acta Histochem. 101, 305 (1999).
Nakamura S. et al., Science 255, 1437 (1992).
Oikawa T et al., Biochem Biophys Res Comm, 246, 243 (1998)
Osman M. et al., J. Virol. 73, 6136 (1999).
Peterson B. A., G. Frizzera, Semin. Oncol. 20, 636 (1993).
Ramsay A. J. et al., Science 264. 561 (1994).
Ribatti, D., et al. Int. J. Dev. Biol. 40, 1189 (1996).
Rizzieri D. A. et al., Lancet 349, 75 (1997).
Samaniego F. et al., J. Immunol. 154, 3582 (1995).
Samaniego F. et al., J. Immunol. 158, 1887 (1997).
Samaniego F. et al.,. Am. J. Pathol. 152, 1433 (1998)
Sgadari C. et al., J. Immunol. 165, 509 (2000).
Siriani M. C. et al., J. Pathol. 152, 1433 (1998)
Siriani M. C. et al., 2^{nd} international workshop on KSHV/HHV8 and related agents.
St. Catherine's College, Oxford, UK. 10-13 September (1999).
Siriani M. C. et al., 3^{rd} international workshop on Kaposi's sarcoma-associated herpesvirus and related agents, Amherst, Mass., USA. June 6-10 (2000).
Stetler-Stevenson W. G., J. Clin. Invest. 103, 1237 (1999).
Taraboletti G. et al., FASEB J. 14, 1674 (2000).
Tosato G. et al., J. Clin. Invest. 91, 2806 (1998).
Tovo P A., AIDS 14, 743 (2000).
Wang Q. J. et al., J. Infect. Dis. 182, 928 (2000).
Weichold F. F. et al., J. Hum. Virol. 2, 261 (1999).

1. Use of at least one compound selected in the group of the inhibitors of the protease of the HIV virus, HIV-PI, for the preparation of a medicament for treating a subject suffering from or susceptible to a condition which can be treated or prevented by blocking the migration/invasion of cells selected in the group of: endothelial, neoplastic, inflammatory or immune cells.

2-26. (canceled)

27. A method for treating a tumor, comprising administering to a first subject having a tumor an HIV protease inhibitor at a daily dose that is lower than the commonly used daily dose of the HIV protease inhibitor administered to treat an HIV infection in an HIV-infected subject.

28. A method for blocking the growth of a tumor, comprising administering to a subject in need of said blocking an HIV protease inhibitor at a daily dose that is lower than the commonly used daily dose of the HIV protease inhibitor administered to treat an HIV infection in an HIV-infected subject.

29. The method of claim 27 or 28, wherein said first subject is not infected with HIV.

30. The method of claim 27 or 28, wherein said HIV protease inhibitor, at a concentration of at least one of 0.1 μM, 1 μM or 10 μM, inhibits endothelial cell and Kaposi's sarcoma cell migration or invasion in response to 50 ng/ml bFGF in a Boyden chamber assay.

31. The method of claim 27 or 28, wherein said HIV protease inhibitor, at a concentration of at least one of 0.1 μM, 1 μM or 10 μM, inhibits the production of cytokines by Kaposi's sarcoma cells.

32. The method of claim 27 or 28, wherein said HIV protease inhibitor, at a dose that corresponds to the whole dose, double, or half the dose of the HIV protease inhibitor used daily in patients infected with HIV, inhibits angiogenesis induced by bFGF in mice.

33. The method of claim 27 or 28, wherein said HIV protease inhibitor, at concentrations of at least one of 0.1 μM, 1 μM or 10 μM, inhibits activation of MMP-2 in cultured human umbilical vein endothelial cells incubated in 0.1 μg/ml bFGF.

34. The method of claim 27 or 28, wherein said HIV protease inhibitor is indinavir or a derivative thereof.

35. The method of claim 34, wherein said daily dose is 600 mg.

36. The method of claim 34, wherein said daily dose is 1200 mg.

37. The method of claim 27 or 28, wherein said HIV protease inhibitor is saquinavir or a derivative thereof.

38. The method of claim 37, wherein said daily dose is 900 mg.

39. The method of claim 37, wherein said daily dose is 1800 mg.

40. The method of claim 27 or 28, wherein said HIV protease inhibitor is selected from the group consisting of indinavir, saquinavir, ritonavir, nelfinavir, amprenavir, and lopinavir; and derivatives thereof; and combinations thereof.

41. The method of claim 40, wherein said HIV protease inhibitor is a combination of indinavir and nelfinavir.

42. The method of claim 27 or 28, wherein the tumor is Kaposi's sarcoma.

43. The method of claim 27 or 28, wherein the tumor is a soft tissue tumor or tumor of cartilage, bone or blood.

44. The method of claim 27 or 28, wherein the tumor is a malignant tumor.

45. The method of claim 27 or 28, wherein tumor is an endothelial cell tumor, a lung cell tumor, a breast cell tumor, a hepatocarcinoma or a leukemia, and in which the first subject is not infected with HIV.

46. The method of claim 27 or 28, wherein the tumor is a myelo-monocytic leukemia or a T cell leukemia, and in which the first subject is not infected with HIV.

47. The method of claim 27 or 28, wherein said administering of the HIV protease inhibitor results in regression of said tumor.

48. The method of claim 27 or 28, wherein said administering of the HIV protease inhibitor inhibits metastasis.

49. The method of claim 27 or 28, wherein said administering of the HIV protease inhibitor further inhibits oedema.

50. The method of claim 27 or 28, wherein the HIV protease inhibitor is administered with an anti-inflammatory, anti-angiogenic or anti-tumor drug.

51. The method of claim 27 or 28, wherein the HIV protease inhibitor is administered orally, intravenously, intramuscularly, subcutaneously, intradermally, intraperitoneally, intrathecally, intrapleurally, intrauterine, transmucosally, rectally, vaginally or percutaneously.

52. The method of claim 27 or 28, wherein the HIV protease inhibitor is administered intralesionally.

53. A method for treating a tumor, an inflammatory disease or an autoimmune disease, comprising administering indinavir or a derivative thereof to a subject in need of said treating.

54. A method for blocking the growth of a tumor, comprising administering indinavir or a derivative thereof to a subject in need of said blocking.

55. The method of claim 53 or 54, wherein said subject is not infected with HIV.

56. The method of claim 53 or 54, wherein the indinavir or derivative thereof is administered at a daily dose that is lower than the commonly used daily dose of indinavir administered to treat an HIV infection in an HIV-infected subject.

57. The method of claim 53 or 54, wherein the indinavir or derivative thereof is administered at a daily dose that is equal to or higher than the commonly used daily dose of indinavir administered to treat an HIV infection in an HIV-infected subject.

58. The method of claim 53 or 54, wherein said daily dose is 2400 mg or 4800 mg.

59. The method of claim 53 for treating a tumor.

60. The method of claim 59, wherein the tumor is Kaposi's sarcoma.

61. The method of claim 59, wherein the tumor is a soft tissue tumor or tumor of cartilage, bone or blood.

62. The method of claim 59, wherein the tumor is a malignant tumor.

63. The method of claim 59, wherein the tumor is an endothelial cell tumor, a lung cell tumor, a breast cell tumor, a hepatocarcinoma or a leukemia, and in which said first subject is not infected with HIV.

64. The method of claim 59, wherein the tumor is a myelo-monocytic leukemia or a T cell leukemia, and in which said first subject is not infected with HIV.

65. The method of claim 59, wherein said administering of the HIV protease inhibitor results in regression of said tumor.

66. The method of claim 59, wherein said administering of the HIV protease inhibitor inhibits metastasis.

67. The method of claim 59, wherein said administering of the HIV protease inhibitor further inhibits oedema.

68. The method of claim 53 for treating an inflammatory disease.

69. The method of claim 68, wherein the inflammatory disease is a chronic inflammation associated with allergies and with viral infective, bacterial or parasitic agents, or the Castleman's multicentric disease.

70. The method of claim 53 for treating an autoimmune disease.

71. The method of claim 70, wherein the autoimmune disease is systemic lupus erythematosus, scleroderma, rheumatoid arthritis, psoriasis, thyroiditis, ulcerous rectocolitis, Crohn's disease, Goodpasture's syndrome, systemic vasculitis, Sjogren's syndrome, or primitive biliary cirrhosis.

72. The method of claim 53 or 54 further comprising administering nelfinavir to the subject.

73. The method of claim 53 or 54, wherein the HIV protease inhibitor is administered with an anti-inflammatory, anti-angiogenic or anti-tumor drug.

74. The method of claim 53 or 54, wherein the HIV protease inhibitor is administered orally, intravenously, intramuscularly, subcutaneously, intradermally, intraperitoneally, intrathecally, intrapleurally, intrauterine, transmucosally, rectally, vaginally or percutaneously.

75. The method of claim 53 or 54, wherein the HIV protease inhibitor is administered intralesionally.
